(19) United States
(12) Patent Application Publication
(10) Pub. No.: US 2012/0308635 A1
(43) Pub. Date: Dec. 6, 2012

(54) MELT EXTRUDED THIN STRIPS CONTAINING COATED PHARMACEUTICAL

(75) Inventors: Caroline Bruce, Boulder, CO (US); Mark Manning, Lincoln, NE (US)

(73) Assignee: Novartis AG, Basel (CH)

(21) Appl. No.: 13/519,915

(22) PCT Filed: Dec. 30, 2009
(86) PCT No.: PCT/US09/69793

§ 371 Date: Jun. 29, 2012

(51) Int. Cl. A61K 9/70; A61P 11/14; A61K 31/4748

(52) U.S. Cl. 424/443; 514/289

(57) ABSTRACT

A composition suitable for hot melt extrusion to form thin strips containing active pharmaceutical ingredients is provided. The composition has 10 to 75% by weight of polyethylene oxide having a molecular weight of from 70,000 to 230,000 Daltons; 5 to 35% of a sugar alcohol having a melting point in excess of 75° C.; 5 to 20% by weight of polyethylene glycol having a molecular weight of from 100 to 4,000 Daltons; and 10 to 75% by weight of coated active pharmaceutical ingredient (API).

### BACKGROUND OF THE INVENTION

This application relates to melt extruded thin strips containing an active pharmaceutical ingredient (API) in coated granular form. The thin strips quickly dissolve in the mouth for passing coated pharmaceutical active through the oral mucosa for absorption in the stomach and/or intestine.

Edible films or films that dissolve in the mouth have been used in a variety of applications, including drug and vitamin delivery and delivery of breath freshener. (See U.S. Pat. Nos. 5,948,430, 6,596,298 and U.S. Pat. No. 6,923,981 which are incorporated herein by reference). Such films are commonly made by a wet casting process. (See U.S. Pat. No. 7,425,292 which is incorporated herein by reference). It has also been suggested that extrusion techniques may be employed (see U.S. Pat. Nos. RE33,093, 6,072,100, and 6,375,963 which are incorporated herein by reference). The selection of materials to be used in a thin strip as well as the most suitable manufacturing approach for the thin strip are dependent on, inter alia, the API to be included in the strip, the API concentration, and the requirements for its delivery. For example, where the API is intended to be delivered in the stomach or intestine, rapid disintegration and passage through the mouth are desired. Conversely, in cases where the delivery desired is a transmucosal delivery in the mouth (for example transbuccal), a much slower disintegration time is desired. In addition, the strip must have the ability to carry (and then release) a sufficient amount of the API, and the API must not be damaged or destroyed in the manufacturing process.

Controlled delivery of drugs frequently involves the use of coatings to impart taste-masking the API, acid- or enzyme-resistance, delayed release, and other desirable release properties. A preferred method of employing such coatings is to directly coat a granulation of the desired pharmaceutical active ingredient. Such granules can be almost entirely active drug, or can be built up from seeds, or by other techniques readily familiar to those of skill in the pharmaceutical manufacturing arts. U.S. Pat. No. 5,009,892, which is incorporated herein by reference, discloses coated granules that can be compressed into tablet form oral consumption. Coated granules are suitable for delivering an API quickly through the mouth past the oral mucosa for absorption of the API in the stomach and/or intestine.

It has proven difficult to obtain a quick-dissolve melt extruded film containing coated pharmaceutical granules which have acceptable properties. There is a need for such quick dissolving melt extruded films containing coated pharmaceutical granules.

### SUMMARY OF THE INVENTION

The present Inventors have herein found that the compositions of the present invention are able to be melt extruded into thin films having preferable properties. In particular the Inventors have unexpectedly found that the compositions of the present invention can be melt extruded under mild conditions (e.g. at a low temperature and low extruder screw speeds) thereby preventing degradation of the coating or API of coated API granules and thus preserving the taste-masking/controlled-release properties of the coated API. Furthermore, the Inventors have found that the thin strips formed from these compositions contain sufficient API loading and are quick to dissolve in the mouth for passing the API to the stomach and/or intestine for delivery.

In a first embodiment, the present invention provides a orally-dissolving pharmaceutical-containing thin strip: 10 to 75% by weight of polyethylene oxide having a molecular weight of from 70,000 to 230,000 Daltons; 5 to 35% of a sugar alcohol having a melting point in excess of 75° C.; 5 to 20% by weight of polyethylene glycol having a molecular weight of from 100 to 4,000 Daltons; and 5 to 75% by weight of coated active pharmaceutical ingredient (API).

In a second embodiment the present invention provides a method of foaming a thin strip comprising the steps of: (I) forming the composition described above; (II) hot melt extruding a thin sheet from the composition; and (III) cutting the thin sheet into thin strips; wherein the processing temperature during steps (I), (II), and (III) does not exceed the melting point temperature of the sugar alcohol.

### BRIEF DESCRIPTION OF THE DRAWINGS

FIG. 1 shows graphical results of Examples 1-5.

FIG. 2 shows graphical results of Examples 1-5.

FIG. 3 shows graphical results of Examples 6-17.

FIG. 4 shows graphical results of Examples 21-30.

FIG. 5 shows graphical results of Example 36.

FIG. 6 shows graphical results of Example 37.

FIG. 7 shows graphical results of Example 36.

FIG. 8 shows graphical results of Example 37.

FIGS. 9 though 11 show graphical results of Examples 38-51.

FIGS. 12 though 14 show graphical results of Examples 52-58.

FIGS. 15 though 23 show graphical results of Examples 59-66B.

FIGS. 24 through 26 show graphical results of Illustration 8.

FIGS. 27 through 32 show graphical results of Illustration 9.

### DETAILED DESCRIPTION OF THE INVENTION

Numerical values in the specification and claims of this application, particularly as they relate to polymeric materials, reflect average values for a composition that may contain individual polymer molecules of different characteristics. Furthermore, the numerical values should be understood to include numerical values which are the same when reduced to the same number of significant figures and numerical values which differ from the stated value by less than the experimental error of the measurement technique used to determine the value.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) may be combined in any suitable manner in the various embodiments.

In order to provide an acceptable thin strip containing a coated API, a strip formulation and production process will desirably have several important characteristics, including inter alia:

(1) The strip formulation has the ability to carry sufficient amount of API to provide a desired dose of API in a strip of a size considered acceptable to a user. Strips that have too little carrying capacity require too large a strip, or the use of too many strips to be considered acceptable by the consumer.

(2) A strip dissolution time in the mouth that is appropriate to the deliver the API through the oral mucosa into the stomach or beyond for dispersion and absorption. Too long of a dissolution time results in the API being dispersed in the mouth leading to unpleasant taste or improper absorption location. A strip dissolution time of less than one or two minutes (e.g. about thirty to 45 seconds or less) is often preferred.

(3) The capability of being formed into a thin strip without substantial degradation of the coating and/or API in the original formulation.

(4) The API contained in the film should not substantially degrade over time. Furthermore, the thin film should have a suitable shelf life so that it can be manufactured, transported, and sold to a consumer while maintaining the desirable properties described herein.

Thin strips can be formed by solvent casting techniques where strip ingredients including the API are dissolved or suspended in a carrier solvent. The slurry or solution is then applied to a sheet, or some other surface, having a large surface area where the solvent is driven off from the solution leaving the desired ingredients in thin film form. The solvent casting process is run in a batch mode and requires several pieces of processing equipment including those that deal with solvent recapture and purification. This approach has been found not to be particularly suitable for forming thin strips containing coated active pharmaceutical ingredients (API). In this regard it has been found that thin strips formed by the solvent casting approach are often to thin to contain desired loading of the API. It has further been found that during the solvent casting approach interaction between the solvent and the coating of the API and in some cases with the API itself may occur. This has been found to be disadvantageous in that thinning of the coating decreases the intended purpose thereof (e.g. controlled release/taste masking). Furthermore, the API can degrade if exposed to solvents (or other compounds in solution or ambient thereto) thereby decreasing the effective active dosage concentration within the thin strip. Lastly, during the expensive and energy-intensive “drying” phase of the solvent casting approach API may also be removed with the solvent, thereby also decreasing the effective dosage concentration within the thin strip during formation.

Thin strips can also be formed by a hot melt extrusion process whereby ingredients are combined in, or prior to introduction to, an extruder which heats and mixes the ingredients and melt extrudes a laminar composition which is then calendered and cut/punched to provide thin strips of desired thickness. While a hot melt extrusion process can be run in continuous or semi-continuous modes, prior hot melt extrusion processes and extrusion formulations have been found not particularly suitable for producing acceptable thin strips containing coated API. In particular, throughout development of processes and formulations suitable for producing coated API containing thin strips, the present Inventors found that process parameters including extruder operating temperature, shear, pressure, screw speed, and flow rate inter alia can lead to degradation of the coating material and of the API. The Inventors also found to their surprise that compositions they initially believed to be suitable for melt extruding into acceptable thin strips were in fact not compatible with extrusion processes and/or exhibited undesirable properties when in film form.

### The Melt Extrusion Composition:

The present invention provides a coated API containing composition suitable for extrusion to produce thin strips. The composition allows for the formulation of thin strips that achieve the properties described above. In particular thin strips made from the present composition have the ability to carry a sufficient amount of coated API to provide a desired dose of API in a strip of a size considered acceptable to a user. The strip dissolution time in the mouth is appropriate to deliver the API through the oral mucosa into the stomach or beyond for dispersion and absorption without unpleasant taste or unintended API absorption therein.

The composition of the present invention comprises polyethylene oxide; a sugar alcohol, having a melting point in excess of 75° C.; low molecular weight polyethylene glycol or a similar plasticizer; and coated API. In a first embodiment, the composition comprises: 10 to 75% by weight of polyethylene oxide having a molecular weight of from 70,000 to 230,000 Daltons; 5 to 35% of a sugar alcohol having a melting point in excess of 75° C.; 5 to 20% by weight of polyethylene glycol having a molecular weight of from 100 to 4,000 Daltons; and 5 to 75% by weight of coated active pharmaceutical ingredient (API).

The term “coated API” refers to API that is coated while in granular and/or pre-dosage form. “Coated API” does not refer to coated dosage size tablets of compressed API that is subsequently coated. The type of coating and API selected for the coated API of the present invention are likewise not particularly limited and such coated API and methods of coating are well known in the art. The combination and total amount of coated granular or pre-dosage API in the thin strip forms the actual dose ingested by the user. In a preferred embodiment, the coated API is in granular form, where the average granule size is between 20 microns to 600 microns, for example between 50 microns to 400 microns, more preferably between 80 microns and 200 microns (e.g about 100 microns). The size of the coated API may be varied to achieve preferred organoleptic properties for the thin strip. In general, the API granules should have a particle size distribution such that not too many API particles are greater than a certain size to prevent the film from tasting gritty before or after film disintegration. It is also preferred that not too many of the API particles be too small because this can cause problems such as dust formation and difficulty of achieving uniform particle size distribution in the films.

In one embodiment, the coating material for the API is selected for the purpose of taste masking. In other embodiments the coating material is selected for controlled or targeted delivery of the API within a user's digestive system. In most preferred embodiments the API in the thin strip will include an over-the-counter API. Such over-the-counter APIs are well known in the art and include analgesics, antihistamines, antitussives (e.g. dextromethorphan HBR), anti-inflammatories, expectorants, upper and lower GI active ingredients, and smoking cessation active ingredients among many other over-the-counter APIs. In other preferred embodiments, the API in the thin strip will be available only by prescription.

The coating material is not particularly limited and may be selected from those well-known in the art. The coating material is selected such that it will withstand the time at temperature and the shear forces imposed by the extrusion process. In other words the coating is selected such that the thermal history of the thin strip formation process is not high enough to degrade the coating. Preferably the coating material will have a melting point above the melt temperature and set point temperatures incurred in the processing equipment (e.g. the hot melt extruder and the calendering rolls). In the embodiments where the coated API is introduced to the hot melt extruder in a downstream barrel toward the composition exit port, the coating material may be selected such that the remaining residence time and melt temperature of the composition in the extruder is such that the coating material is not degraded. In preferred embodiments the coating material will have a melting point temperature (Tm) at least 5° C., 10° C., 20° C., 30° C., 40° C. or more below the maximum temperature it will encounter during the extrusion and calendering processes described herein.

In some embodiments the coating material is a polymeric material that requires a specific pH range to initiate dissolution thereof (e.g. the pH range of the stomach or pH range of the intestine). In other embodiments the coating material selected from the group consisting of: ethyl cellulose and cellulose acetate.

The coated API will be present in the formulation in an amount sufficient to provide a desired and/or suggested dose of the API in a thin strip or combination of thin strips. In particularly preferred embodiments, the coated API will make up 5 to 75% by weight of formulation, more preferably between 10 wt %, or 25 to 65 wt % of the formulation, like between 28 to 32 wt % (e.g. 30 wt %) of the formulation.

Polyethylene oxide (PEO) suitable for use in the compositions of the present invention has a weight average molecular weight (Mw) of from 70,000 to 230,000, more preferably 85,000 to 215,000 (e.g. about 100,000) Daltons. Significantly higher molecular weights, or compositions that include coagulants that cause an increase in molecular weight of the polyethylene oxide are generally not desired. PEO with these characteristics is available from Dow Chemical as POLYOX™ WSR N-10 (Mw about 100,000 Daltons) and POLYOX™ WSR N-80 (Mw about 200,000 Daltons). Of these, POLYOX™ WSR N-10 is frequently preferred.

The PEO is suitably present in the composition of the invention in an amount of 10 to 75 weight %, more preferably between 25 and 45 wt %, and most preferably between 25 to 35% (e.g. 30 wt %) of the formulation. It is noted that PEO is also referred to in the art as polyethylene glycol (PEG). However, since a low molecular weight plasticizer, that may be PEG, is also used in the composition this component is referred to as PEO to maintain a distinction.

The compositions of the invention also include a low molecular weight plasticizer. Such plasticizers include glycerin, propylene glycol, Triethyl citrate, and polyethylene glycol (PEG). In a preferred embodiment the low molecular weight plasticizer is PEG, which is miscible with PEO, having a weight average molecular weight (Mw) of between 100 and 4000 Daltons, more preferably between 300 and 500 Daltons (e.g. 400 Daltons or PEG 400 in liquid form). The PEG is present in an amount of 5 to 20 wt % of the formulation, more preferably between 7 and 15 wt % (e.g. 10 wt %) of the formulation.

The composition of the present invention also contains a water-soluble polyol (e.g. a sugar alcohol). The polyol is selected to have a melting point that is greater than 75° C., more preferably greater than 90° C., 100° C., 110° C., 130° C., or greater than 150° C. The polyol is preferably selected such that its melting point is in excess of the highest temperature at which the formulation will be treated during formation of thin strips. Without intending to be bound by any particular mechanism, it is believed that sugar alcohols are soluble in water and saliva and are effective to enhance the dissolution rate of the thin strips molded from the composition, with higher levels of sugar alcohol resulting in more rapid dissolution. It is believed that the sugar alcohol dissolves quickly creating a porous matrix in the thin strips for rapid dissolution of the other components. Thus, increased levels of sugar alcohol may be used to offset higher molecular weight PEO. In general, sugar alcohol levels of 5 to 35 weight % of the composition, more preferably between 15 and 30 wt % (e.g. 22.8 wt % or 30 wt %) of the composition.

Specific and non-limiting examples of sugar alcohols useful for this purpose include sorbitol, xylitol, mannitol, lactitol and maltitol. In other embodiments erythritol may optionally be used as the sugar alcohol or in combination with other sugar alcohols. Of these sorbitol (melting point 95° C.) and mannitol (melting point 167° C.) are particularly preferred, with mannitol being most preferred.

The art of adding other excipients to pharmaceutical preparation is well known in the art and such additions do not depart from the scope of the present invention. For example the compositions of the present invention may be blended with well-known flavoring compositions containing active flavorants to form a flavored blend suitable for hot melt extrusion to form thin strips. A non-limiting list of exemplary active flavorants include capsaicin, pieprine, chavicine, vanillin, vanillyl butyl ether, vanillyl ethyl ether, N-nonanoyl vanillylamide, gingerols, zingerone, and combinations of other natural and artificial flavors such as orange, grape, vanilla, cherry, grape, cranberry, peppermint, spearmint, anise, blueberry raspberry, banana, chocolate, caramel, citrus, strawberry, lemon, and lime. These active flavorants are often blended with a bulk carrier to form a flavoring composition for more efficient and even distribution within the presently contemplated composition. Typically, where a flavoring composition (e.g. an active flavor disposed in a bulk carrier) is used it will make up about 2 to 20 wt % of the thin strip. Other additives (e.g. sweetners and preservatives) are well known in the art and may optionally be blended with the composition.

### The Holt Melt Extrusion Process:

The Inventors have quite surprisingly found that the thin strip formulation of the present invention allows for treatment under mild process conditions (e.g. low temperature, shear, and pressure, inter alia). The thin strip formulation can be melt extruded at melt temperatures below 150° C. (e.g. below 90° C., 80° C., 70° C., 60° C., and in some embodiments even below 50° C., for example at 40° C. or 45° C.). Due to frictional stresses incurred within the extruder the melt temperature of components is often a few degrees more than the set point temperature of the extruder. Therefore, care should be taken to ensure the melt temperature of the components within the extruder is within these ranges. Furthermore, it is preferred to select an extruder screw speed and throughput flow rate where frictional temperature gains within the extruder are minimized. As described herein, frictional stresses upon the composition can also lead to leaching of the API from through the coating material. Treating the thin strip formulation at these mild process conditions allows for preservation of the coating material as well as preservation of the API. The invention therefore also provides a method of forming a thin strip at these mild process conditions as well as thin strips formed at these mild conditions.

The hot melt extrusion composition of the present invention may be formed prior to introduction to the extruder or within the extruder itself. Where the composition is formed prior to introduction to the extruder it is preferred that the temperature profile of the extruder and subsequent processes (e.g. calendering) be maintained at a temperature of less than the melting point of the sugar alcohol or the sugar alcohol and the coating material of the API. Where the sugar alcohol is mannitol this temperature should be less than 150° C. Where the sugar alcohol is sorbitol this temperature should be less than 90° C. In most preferred embodiments this temperature will be between 50 and 70° C. to prevent melting of the sugar alcohol and degradation of the coating material and the API itself.

Where the composition is formed within the extruder (e.g. by side stuffing one or more of the components) certain portions of the extruder may be operated at temperatures greater than those described above, thereby treating some of the components of the composition at elevated temperature for extended periods of time. In the later embodiment it is again preferred to minimize exposure of the API to elevated temperature for an extended period of time. Therefore, in another preferred embodiment the coated API is introduced/side-stuffed to the extruder in a downstream barrel section from where other components are introduced. For example a portion or all of the coated API is side-stuffed into the extruder and the extrusion composition is formed and thoroughly mixed by the time the barrel exit section (e.g. the die) of the extruder is reached. The upstream barrel sections from the API side stuffing barrels) may be operated at elevated temperatures. The side-stuffing barrel section and downstream barrel sections are preferably operated under the preferred temperatures ranges described above.

Upon exit from the extruder the extrudate can be calendered to its desired thickness using one or more optionally temperature-controlled calendering rolls. Where the rolls are temperature controlled, it is preferred to select a temperature where the extrudate does not stick to the rolls. The controlled roll temperature can be for example between 10° C. and 100° C., more preferably between 20 and 70° C., for example between 25 and 50° C. (e.g. 30° C.). In preferred embodiments the thickness of the thin strip is between 0.05 mm and 2 mm, for example between 0.1 mm and 0.8 mm (e.g. between 0.2 mm and 0.5 mm). In other preferred embodiments the thickness of the thin strip is less than 0.4 mm, for example 0.3 mm or 0.25 mm.

The calendered composition can then be introduced to a backing material and then rolled to form a master roll. The master roll then can be cut into feeder rolls having the desired thin strip width or length and then unwound and cut or scribed to form dosage size thin strips. The amount of coated API in the thin strip will be a function of the size of the thin strip (length×width×thickness) and the concentration of the API in the composition. In a preferred embodiment, an individual thin strip will contain a recommended dose of the API. In preferred embodiments, the thin strip will be from 0.5 to 4 cm wide by 0.5 to 6 cm long. In other embodiments the thin strip will be from 1.5 to 3 cm wide (e.g. about 2 cm wide) by 1.5 to 5 cm long (e.g. about 3.5 cm long). Once in individual thin strip form the strips may be individually packaged or combined with others and packed in a multiple dose container (e.g. in ribbon/dispenser for stacked form).

As described herein, the formulation and techniques of the present invention allow for the preparation of thin strips compositions containing coated API. The processes described allow for the preservation of the coating material so as to prevent seepage of the API into the surrounding composition (e.g. free API). The amount of seepage of the API from the Coated API during the formation of the thin strip can be determined by comparing the content of free API in an unprocessed amount of coated API to the same amount that should be in a formed thin strip. One method described below for accomplishing this is to determine a solvent where the API and other thin strip components are dissolvable therein but the coating material is not. A specified amount of the coated API is then placed in the solvent for a specified time (e.g. 2 minutes) and the amount of free API in the solvent in determined. Next, a thin strip of which size and concentration should contain the same amount of coated API is placed in the solvent for the specified time and the free API is also determined. The two values are compared to determine how much seepage of API from the coated API occurred during the thin strip formation process. In most preferred embodiment the thin strip formation methods of the present invention will produce a thin strip that contains less than five times (e.g. less than 3 times, less than 2 times, and most preferably less than 1.5 times) the amount of free API compared to a corresponding amount of unprocessed coated API used in the preparation.

### EXAMPLES

Having described the invention in detail, the following examples are provided. The following examples provide acceptable and preferred strategies of forming test strips that are acceptable for use in industry. The examples should not be considered as limiting the scope of the invention, but merely as illustrative and representative thereof.

The terms “working” and “comparative” are simply used to demonstrate comparisons to other examples. A comparative example may or may not be an example within the scope of the present invention.

The present Inventors have found that the composition listed in Table 1A is superior for use in extrusion processes for forming thin films containing coated API. Table 1B lists a more preferred composition according to one embodiment. The present Inventors have quite unexpectedly found that the present composition may be processed at mild conditions (e.g. low shear and more importantly at low temperature) to form coated API-containing thin strips with superior properties.

TABLE 1A
preferred thin strip extrusion composition of the present invention.

| Component | % wt |
|---|---|
| Coated API granules | 5-75 |
| PEO, MW 70k-230k Dalton | 10-75 |
| Sugar Alcohol having a MP >75 C. | 5-35 (e.g. 30) |
| PEG, MW 100-4000 Dalton | 5-20 (e.g. 10) |

TABLE 1B
more preferred thin strip extrusion composition of the present invention.

| Component | % wt |
|---|---|
| Coated API granules | 25-65 (e.g. 30) |
| PEO, MW 70k-130k Dalton (e.g 100k Dalton) | 25-45 (e.g. 30) |
| Sugar Alcohol having a MP >75 C. (e.g. Mannitol) | 15-30 (e.g. 30) |
| PEG, MW 100-1000 Dalton (e.g 400 Dalton) | 7-15 (e.g. 10) |

Preferably the thin strip is between 0.05 millimeters and 2.00 millimeters thick.

Several additives such as preserving, coloring, and flavoring agents, inter alia, are known in the art and may be combined with the composition (see Illustration 7). The addition of additives does not depart from the scope of the present invention.

The following Illustrations are provided to demonstrate how to make, melt extrude, and form thin strips from the composition of the present invention. They also demonstrate the unexpected ability to use this composition in a mild extrusion processes (e.g. low temperature and low shear) and the superior properties of the films formed using these compositions at mild conditions. Although the following examples show the use of taste-masked coated Dextromethorphan HBr, it will be appreciated by those skilled in the art that other coated APIs may be used in association with the formulation of the present invention.

### Introduction to Illustrations.

The following list of compositions in Table 2 are ones that were believed to potentially be able to form melt extrudable fast-dissolving films.

TABLE 2
Polymers investigated in this study

Potential Film-Forming Polymers
Starch
Polyethylene oxide
Hydroxypropyl cellulose
Kollicoat IR

Each of these compositions was melt extruded to determine whether such composition possessed desirable properties. The objective of the initial extrusions was to survey these polymers in combination with plasticizers and/or secondary polymers to find combinations that could be used in further development.

Three criteria were observed:

1. Could the powder blend be extruded by melt extrusion equipment?
2. Could a film be calendared? A film could be calendared if the film was not too sticky, and flexible enough to be formed by the rolls.
3. Did the film disintegrate quickly?

The following descriptions are short summaries for each of the polymers tested.

### Polyethylene Oxide (PEO)

This material (Mw 100,000 Dalton) was found to be a good basis for thin film production as it was extrudable, not significantly tacky, and had desired disintegration properties. The illustrations provided below include further examples with polymer.

### Starch

Starch was considered a good choice as the material is a natural material widely used for extrusion processes in the food industry, and is available in a multitude of grades for different applications. Starch 1500, a partially pre-gelatinized starch, was used for initial extrusions. Initially, no viable film was obtained due to insufficient plasticization of the material. Plasticizer was added in sufficient amounts. Thin films with fast disintegration times were obtained, but had undesirable film properties, such as stickiness and brittleness. The illustrations provided below include further examples with polymer.

### Hydroxypropyl Cellulose (HPC)

Several combinations of HPC and different plasticizers were extruded, but none of the films prepared showed desirable properties, being either too brittle, too weak, or having a long disintegration times. This polymer was not tested further.

### Kollicoat IR®

Kollicoat IRO is a polyvinyl alcohol-polyethylene glycol graft copolymer made by BASF. Initial extrusions of formulations failed as no processing temperature window could be found. At low temperatures, insufficient softening occurred despite attempts to plasticize the material, and excessive browning product resulted at slightly higher temperatures. This polymer was not tested further.

### Illustration 1: Extruding Starch-Containing Films (Comparative)

### 1.1 Introduction

These examples show starches capable of being melt-extruded into thin films. Specific formulations were selected with a view of obtaining thin films having acceptable properties. However, the starch extrusions shown below were observed to have undesirable properties for forming fast dissolving API-containing undesirable properties.

### 1.2 Materials and Methods

The following materials of Table 1 were used in preparing the powder blends for melt extrusion.

TABLE 3

| Group | Material | Description |
|---|---|---|
| Starches | Lab 3455 | hydroxypropylated starch, pregelatinized |
| | Lycoat RS 720 | hydroxypropylated starch, higher viscosity |
| | Lycoat RS 780 | hydroxypropylated starch, lower viscosity |
| | Lycoat NG 73 | hydroxypropylated starch, pregelatinized |
| | Nutriose FM06 | Maize dextrin soluble fiber |
| | Lycatab PGS | completely pregelatinized starch |
| | Powdered 400L | modified corn starch |
| Sugar | Xylisorb 300 | Xylitol |
| Alcohols | Neosorb P110 | Sorbitol |
| | Pearlitol 100SD | Mannitol |
| Others | Glycerol | |
| | Gel-Klear 085 | Gelatin, PEG monoglycerides, |
| | | glycerin, maltodextrin, SiO₂ |
| | TEC | Triethylcitrate |
| | Precirol ATO5 | Glycerol distearate |
| | Syloid 244 FP | Silicon dioxide |
| | Citric acid | Anhydrous grade |
| Drug | Dextrome- | coated and taste masked |
| | thorphan HBr | |

All powders were blended in a high-shear mixer. Liquid components were added to granulate the powders, and the blend was transferred to the hopper of a K-Tron gravimetric feeder. Blends were extruded on a counter-rotating twin-screw Leistritz ZSE-18 (diameter 18 mm, barrel length 40 D) through a film die. Extrusion temperatures varied from 80 to 100° C.

Samples were collected of all compositions which could be extruded. Initial descriptions of films were noted as the formulations were being extruded.

Three days after the end of the extrusion run, all films were handled to observe appearance, tackiness, and flexibility or rigidity of the materials. The films were sorted into three categories: Films that are too tacky (six formulations), films that are too rigid (six formulations), and films with acceptable properties (five formulations).

Disintegration testing was performed on the films with acceptable properties. Film samples were cut with a stainless steel punch, slipped into a paperclip (used as a sinker), and placed into a USP disintegration testing apparatus. The test was performed in triplicate in deionized (DI) water at 37.0±0.3° C., and was timed with a stop watch.

### 1.3.1 Results

Tables 4 to 6 list the compositions of the films with acceptable processing properties. All films were extruded at 90° C. Films with acceptable properties contained Lycatab PGS (completely pre-gelatinized starch), Lycoat RS 720 (hydroxypropylated starch, higher viscosity) or Lab 3544 (pre-gelatinized hydroxypropylated starch).

TABLE 4
Films containing Lycatab PGS (completely pre-gelatinized starch)

Example No.

| | 1 | 2 |
|---|---|---|
| Lycatab PGS | 38 | 35 |
| Xylitol | 10 | |
| Sorbitol | | 20 |
| Glycerol | 10 | 10 |
| Gelatin | 2 | |
| Dextromethorphan HBr | 40 | 35 |

TABLE 5
Films containing Lycoat RS 720
(hydroxypropylated starch, higher viscosity)

| | Example No |
|---|---|
| | 3 |
| Lycoat RS 720 | 32 |
| Sorbitol | 18 |
| Glycerol | 5 |
| Talc | 5 |
| Dextromethorphan HBr | 40 |

TABLE 6
Films containing Lab 3544 (pre-gelatinized hydroxypropylated starch)

Example No.

| | 4 | 5 |
|---|---|---|
| lab 3544 | 40 | 35 |
| sorbitol | 20 | 15 |
| glycerol | 10 | 5 |
| Dextromethorphan HBr | 30 | 35 |
| Citric acid | | 10 |

### 1.3.2 Disintegration Times

Table 7 shows the average thickness and disintegration times of the starch-containing films with acceptable processing properties. The disintegration time of a film is affected by its thickness, which should be taken into consideration when comparing disintegration times. Disintegration times, taking into consideration thickness, are slower than desired.

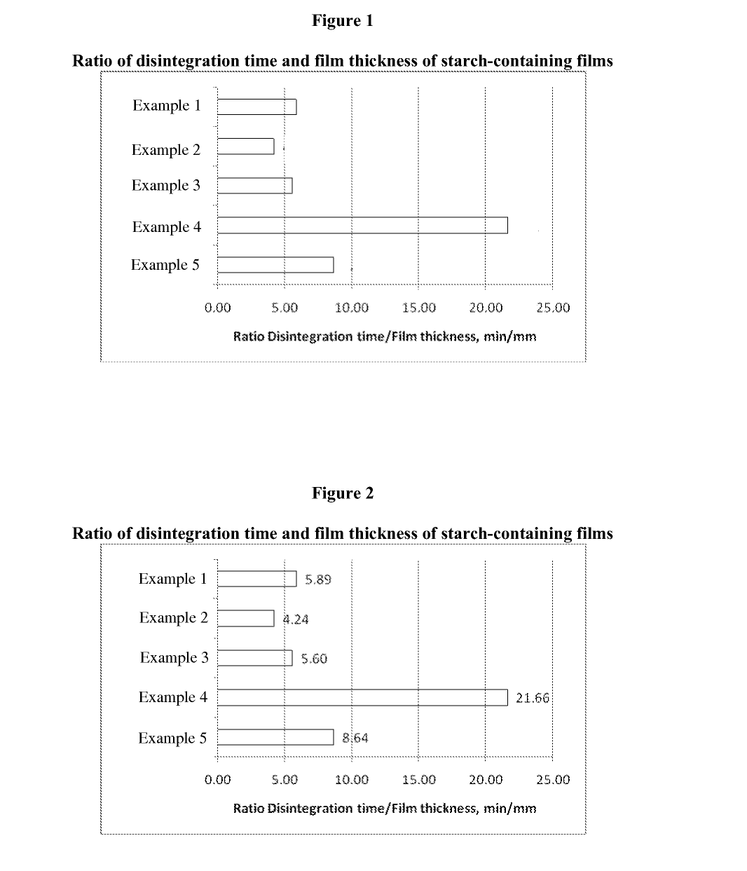

The ratio of disintegration time and film thickness of starch-containing films was calculated to compare disintegration times of films with differing thicknesses (FIGS. 1 and 2). This ratio should be treated with caution, as the relation of disintegration times to thickness are likely not linear, but this approach generates a single metric of comparison. It will be used to identify formulation approaches which have a good probability of fast disintegration.

TABLE 7
Thickness and disintegration times of films with acceptable properties.

| Example No. | Average Thickness, mm | Disintegration Time, min |
|---|---|---|
| 1 | 0.385 | 3.33 |
| 2 | 0.577 | 12.50 |
| 3 | 0.581 | 3.25 |
| 4 | 0.841 | 3.57 |
| 5 | 0.722 | 4.25 |

### 1.4 Conclusions

The mechanical properties of films were used as an initial guidance to judge viability of formulations. Lycatab PGS (completely pre-gelatinized starch), Lycoat RS 720 (hydroxypropylated starch, higher viscosity) or Lab 3544 (pre-gelatinized hydroxypropylated starch) yielded films with acceptable properties under the extrusion conditions.

Glycerol and polyols were adequate plasticizers for starches.

Disintegration times are longer than desired. Modifying the formulations to decrease disintegration times will be investigated in the next course of extrusions utilizing a single starch.

### 2 Extrusion of Films Containing a Hydroxypropylated Starch, Lycoat RS 720 (Comparative)

### 2.1 Introduction

The objective of this work was to use the hydroxypropylated starch Lycoat RS720 to formulate thin, fast-dissolving films containing taste-masked Dextromethorphan hydrobromide.

### 2.2 Materials and Methods

Formulations are listed in Table 8 (.1, .2, and .3). To make the powder blends, dry materials, including the coated API, were weighed into a plastic bag, and mixed by shaking. The liquid components were introduced to the powder using a high-shear granulator (Robot Coupe), before loading the blend into the extruder's gravimetric feeder. Talc and silicon dioxide were added after the wet granulation process and mixed by shaking.

A Leistritz ZSE 18 HP twin-screw extruder equipped with a K-tron gravimetric feeder and a film die was used to extrude the formulations. Feed rate and screw speed were kept constant for the duration of the experiments at lkg/hr and 125 RPM, respectively. The maximum extruder temperatures varied between 80 and 95° C., depending on operator observations.

Films were evaluated immediately after extrusion, and two days after the process. Tackiness, flexibility and brittleness were noted. For disintegration testing, a punch and mallet were used to obtain samples of uniform size, and to standardize the handling of films. The thickness of the punched samples was measured by digital calipers (Mitutoyo), and the samples were slid into paper clips used as sinkers for the test. The disintegration test was performed on a USP disintegration tester (PharmaAlliance), in DI water at 37.2° C.±0.5° C. (n=3).

TABLE 8.1
Formulation compositions

| | Example No. | | | | | | | | | | | |
|---|---|---|---|---|---|---|---|---|---|---|---|---|
| Components, % | 6 | 7z | 8 | 9 | 10 | 11 | 12 | 13 | 14 | 15z | 16z | 17z |
| Lycoat RS720 | 40 | 28 | 30 | 30 | 30 | 30 | 30 | 40 | 35 | 30 | 25 | 25 |
| Coated API | 30 | 40 | 40 | 40 | 40 | 40 | 40 | 25 | 30 | 40 | 40 | 40 |
| Sorbitol | 20 | 20 | 15 | 10 | 10 | 10 | 10* | 10 | 10 | 10 | 10 | 8.3 |
| Glycerol | 10 | 10 | 7.5 | 7.5 | 7.5 | 7.5 | — | 7.5 | 7.5 | 7.5 | 7.5 | 6.25 |
| Filler | — | 2 (0) | 7.5 (1) | 5 (1) | 5 (2) | 5 (3) | 5 (1) | 5 (1) | 5 (1) | 5 (1) | 5 (1) | 7.95 (1) |
| additional | — | | — | 7.5 (4) | 7.5 (4) | 7.5 (4) | 7.5 (4) | 12.5 (4) | 12.5 (4) | 7.5 (5) | 12.5 (5) | 12.5 (5) |
| Film former | | | | | | | | | | | | |
| Extr. temperature, ° C. | 90 | 80 | 80 | 80 | 80 | 80 | 95 | 95 | 95 | 95 | 95 | 95 |

TABLE 8.2
Formulation compositions (cont.)

| Examle No./ | Example No. | | |
|---|---|---|---|
| Components, % | 18 | 19 | 20 |
| Lycoat RS720 | 25 | 25 | 25 |
| Coated API | 40 | 40 | 40 |
| Sorbitol | 15 | 15 | 15 |
| Glycerol | 7.5 | 9 | 7.5 |
| Filler | 2.5 (0) | 2 (0) | 5 (1) |
| additional Film | 10 (5) | 9 (5) | 7.5 (5) |
| former | | | |
| Extr.temperature, ° C. | 80 | 80 | 90 |

(In Tables 8.1 and 8.2—Examples with fastest disintegration times shown in bold and with “z” next to the Example No. Numbers in brackets correspond to the following information).

*—in solution

(0)—colloidal Silicon dioxide

(1)—Talc

(2)—MCC (Avicel PH200)

(3)—Titanium dioxide

(4)—Maltodextrin (Maltrin M180)

(5)—PEG 3350

### 2.3 Results

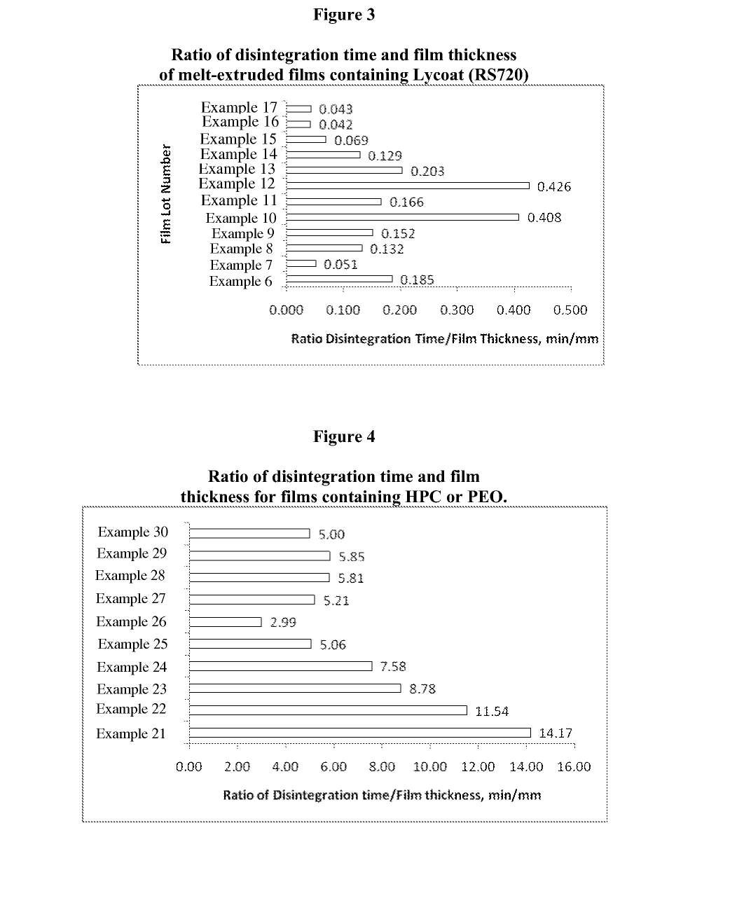

Table 9 lists the disintegration time and film thickness of each formulation. Since the film thickness can affect the disintegration time of a film, the ratio of disintegration time and film thickness was calculated, and is shown in FIG. 3. Macroscopic film properties are described in Table 10.

The strategy of this experimental run was to start with the best-performing films of the last illustration, and then to screen various modifications to the formulation for ease of processing, film properties and disintegration time to improve on it. The ranges of formulation components in well-performing films were used to design the formulation of Example 6, to be used as a starting point. Observations in extrusions then drove further modifications.

Two factors were investigated: the addition of a filler material, enabling the reduction in film-former content, and the introduction of a second film former. Silicon dioxide, talc, microcrystalline cellulose, titanium dioxide were studied as fillers, and maltodextrin and PEG 3350 were used as additional film forming agents with lower molecular weights than the starch. In one instance, a sorbitol-in-water solution was used instead of a sorbitol powder to explore the effect of water in the formulation.

One early formulation (Ex. 7) disintegrated very fast, but its film properties were not conducive to extrusion and handling as it was very tacky.

Films of Exs. 8, 9, 10, and 11 only differed in the type of filler, and the film containing talc could be extruded in a thin film, and showed the fastest disintegration time. Talc was subsequently used as filler.

The type and level of the second film former was investigated in the films of Exs. 13, 14, 15, 16, and 17. In general, films with lower starch content disintegrated faster. Disintegration depends on the wetting, disentanglement and dispersion of film components. A high molecular-weight film former, such as Lycoat RS 720, slows down these processes, compared to formulations in which part of the film forming polymer is replaced by a lower molecular weight material. The addition of PEG 3350 resulted in shorter disintegration times than the addition of Maltodextrin.

Additional extrusions were based on the PEG-containing films to change the brittleness of the films. However, these films were too tacky to be tested further.

TABLE 9.1
Disintegration times and film thickness of melt-extruded films containing Lycoat RS720.

| | Example No. | | | | | | | | | | | |
|---|---|---|---|---|---|---|---|---|---|---|---|---|
| | 6 | 7z | 8 | 9 | 10 | 11 | 12 | 13 | 14 | 15z | 16z | 17z |
| DT* 1, min:sec | 3:56 | 0:23 | 3:47 | 1:30 | 5:10 | 2:33 | 5:12 | 2:21 | 1:39 | 0:27 | 0:20 | 0:25 |
| DT 2 min:sec | 2:02 | 0:33 | 3:04 | 1:41 | 8:08 | 2:30 | 5:52 | 2:45 | 1:25 | 0:23 | 0:17 | 0:19 |
| DT 3 min:sec | 3:31 | 0:41 | 3:53 | 2:22 | 4:33 | 2:16 | 3:41 | 2:40 | 1:17 | 0:30 | 0:14 | 0:15 |
| Average DT | 3:09 | 0:32 | 3:34 | 1:51 | 5:57 | 2:26 | 4:55 | 2:35 | 1:27 | 0:26 | 0:17 | 0:19 |
| Film Thickness 1, mm | 0.736 | 0.381 | 1.171 | 0.508 | 0.635 | 0.662 | 0.500 | 0.52 | 0.507 | 0.253 | 0.288 | 0.301 |
| Film Thickness 2, mm | 0.605 | 0.430 | 1.117 | 0.479 | 0.555 | 0.619 | 0.477 | 0.537 | 0.454 | 0.247 | 0.291 | 0.353 |
| Film Thickness 3, mm | 0.796 | 0.499 | 1.088 | 0.538 | 0.633 | 0.552 | 0.465 | 0.536 | 0.449 | 0.308 | 0.263 | 0.306 |
| Average Thickness | 0.712 | 0.437 | 1.125 | 0.508 | 0.608 | 0.611 | 0.481 | 0.531 | 0.470 | 0.269 | 0.281 | 0.320 |

TABLE 10
Film properties of melt-extruded films containing Lycoat RS720.
*recorded on the day of extrusion only

| Ex. No. | Film properties two days after extrusion |
|---|---|
| 6 | very flexible |
| 7 | very flexible, sticks to itself |
| 8 | very thick, somewhat flexible, tough |
| 9 | flexible until stressed to break |
| 10 | brittle, not sticky |
| 11 | flexible, brittle when stressed, chalky |
| 12 | very brittle, fragile, not tacky |
| 13 | very brittle, breaks on punching, not tacky |
| 14 | very brittle, not tacky |
| 15 | somewhat flexible |
| 16 | somewhat flexible, somewhat brittle, not tacky |
| 17 | very brittle, fragile on punching, not tacky |
| 18 | elastic, very tacky, sticks to calendar rolls and itself |
| 19 | elastic, very tacky, sticks to calendar rolls and itself, |
| | hopper build up |
| 20 | elastic, very tacky, sticks to calendar rolls and itself, |
| | hopper build up |

### 2.4 Summary

Films of Examples 15, 16, and 17 show the fastest disintegration times of extruded films (average of 17 to 26 seconds). These formulations could be formed into relatively thin films by calendaring, but showed tackiness and sticking on the calendar rolls. In addition, after cooling the formulations were brittle, complicating handling. These films were therefore found to have undesirable properties for the required purposes.

3. Disintegration times of films containing HPC and PEO

### 3.1 Introduction

The aim of this study was to screen likely formulations and excipients, and to characterize the disintegration time of the films which were prepared during the first illustration.

### 3.2 Melt Extrusion and Disintegration Testing

Formulations are listed in Table 10. Powder blends (300 g) were prepared by mixing in a plastic bag. Liquid components were added to the blend in a high shear mixer. All formulations were extruded on a Leistritz ZSE-18 (diameter 18 mm, barrel length 40 D) through a film die. Extrusion temperatures varied from 80 to 100° C.

Disintegration testing was performed on the films with acceptable properties. Film samples were cut with a stainless steel punch, slipped into a paperclip (used as a sinker), and placed into a USP disintegration testing apparatus. The test was performed in triplicate in DI water at 37.0±0.3° C., and was timed with a stop watch.

### 3.3 Results

For a given formulation, thicker films have longer disintegration times than thinner films. All thicknesses and disintegration times are listed in Table 10. To be able to compare the propensity of a formulation to disintegrate, the ratio of disintegration time and film thickness was calculated (FIG. 4). This ratio should be treated with caution, as the relation of disintegration times to thickness are likely not linear, but this approach generates a single metric of comparison. It will be used to identify formulation approaches which have a good probability of fast disintegration.

All films discussed in this illustration were screened to be reasonably flexible, strong and non-tacky.

TABLE 11
Composition, average thickness and disintegration times of films
containing HPC or PEO.

| | | | Avg Thickness, | Disintegration |
|---|---|---|---|---|
| Example No. | Component | % w/w | mm | Time, min |
| 21* | Klucel ELF | 45 | 0.406 | 5.75 |
| | Mannitol | 45 | | |
| | TEC | 10 | | |
| 22* | Mannitol | 70 | 0.693 | 8.00 |
| | Klucel ELF | 25 | | |
| | TEC | 5 | | |
| 23* | Klucel ELF | 45 | 0.304 | 2.67 |
| | Xylitol | 45 | | |
| | TEC | 10 | | |
| 24* | PolyOx N10 | 75 | 0.198 | 1.50 |
| | Mannitol | 20 | | |
| | Glycerin | 5 | | |
| 25 | PolyOx N10 | 40 | 0.231 | 1.17 |
| | Coated Dex | 25 | | |
| | Mannitol | 25 | | |
| | PEG 400 | 10 | | |
| 26 | PolyOx N10 | 30 | 0.194 | 0.58 |
| | Coated Dex | 30 | | |
| | Mannitol | 30 | | |
| | PEG 400 | 10 | | |
| 27 | Coated Dex | 35 | 0.225 | 1.17 |
| | PolyOx N10 | 30 | | |
| | Mannitol | 25 | | |
| | PEG 400 | 10 | | |
| 28 | Coated Dex | 35 | 0.258 | 1.50 |
| | Mannitol | 25 | | |
| | PolyOx N10 | 15 | | |
| | Klucel ELF | 15 | | |
| | PEG 400 | 10 | | |
| 29 | Coated Dex | 35 | 0.371 | 2.17 |
| | PolyOx N10 | 15 | | |
| | Klucel ELF | 15 | | |
| | Mannitol | 12.5 | | |
| | Maltrin M180 | 12.5 | | |
| | PEG 400 | 10 | | |
| 30 | Coated Dex | 35 | 0.334 | 1.67 |
| | PolyOx N10 | 30 | | |
| | Mannitol | 15 | | |
| | Maltrin M180 | 15 | | |
| | PEG 400 | 10 | | |

(*These films did not contain API. Presence of the API affected disintegration times)

The presence of API affected film disintegration times due to its form. The granules were unaltered by the melt-extrusion process, and are thought to present weak spots that aid in film disintegration. This property is independent of the API inside the granules. The effect of granule size has yet to be studied.

### 3.4 Summary

Formulations containing polyethylene oxide had the shortest disintegration times and most acceptable film properties.

### 4 Extrusion of PEO and HPC-Containing Films

### 4.1 Introduction

This work concentrated on screening formulations to decrease the disintegration time of thin, melt extruded films.

### 4.2 Formulations

The film of example 26 had the shortest disintegration time, and formed the basis for the formulations extruded in this illustration. Table 11 lists the extruded formulations.

TABLE 12
Formulations.

| | Example No. | | | | |
|---|---|---|---|---|---|
| Components | 31 | 32 | 33 | 34 | 35 |
| Coated API | 30 | 30 | 30 | 30 | 30 |
| Polyox N10 | 30 | 30 | | | 25 |
| Nisso HPC | | | 30 | | |
| Klucel ELF | | | | 30 | |
| Mannitol | 30 | | 30 | 30 | 30 |
| Sorbitol | | 30 | | | |
| PEG 400 | | 10 | 10 | 10 | 10 |
| PEG 3350 | 10 | | | | |
| Nutriose (Maize Dextrin) | | | | | 5 |
| Extrusion temperature, ° C. | 90 | 75 | 80 | 80, 85, 90 | 100 |

### 4.3 Results

Film properties are summarized in Table 12. Disintegration times for all films were slower than in the basic formulation, Example 26.

Film properties were improved in film Example 31, as the higher MW PEG reduced the film tackiness. However, the disintegration time was increased.

The higher solubility of Sorbitol could not be utilized to decrease disintegration time, as the component melted in the process, resulting in poor handling of the resulting film Example 32.

Both films containing HPC were very thick, which disproportionally affected disintegration (Example 33 and Example 34).

The film containing 5% soluble fiber (Example 35) showed a promisingly fast disintegration.

TABLE 13
Properties of extruded films.

| | Example No. | | | | |
|---|---|---|---|---|---|
| | 31 | 32 | 33 | 34 | 35 |
| Average | 0.3737 | 0.5587 | 1.3773 | 1.5297 | 0.2447 |
| Thickness | | | | | |
| (mm) | | | | | |
| SD thickness | 0.0071 | 0.0015 | 0.0142 | 0.0137 | 0.0015 |
| Dis- | 1.83 | 3.00 | 10.50 | 20.00 | 1.00 |
| integration | | | | | |
| Time (min) | | | | | |
| Ratio | 4.90 | 5.37 | 7.62 | 13.07 | 4.09 |
| DT/ | | | | | |
| Thickness | | | | | |
| Comments | calendared | calendared | tacky | tacky | calendared |
| | not tacky | tacky | thick film | thick film, | |
| | | | | DT | |
| | | | | was | |
| | | | | stopped | |
| | | | | after | |
| | | | | 20 min | |

### 4.4 Summary

Of the formulations extruded in this run, Example 35 had the shortest disintegration time. Overall, the PEO formulation 26 is the fastest-dissolving film. The following examples will focus on a further reduction in disintegration time, using this formulation as a starting point.

5 Investigation of the Extrusion Temperature and Screw Speed for a PEO-Containing formulation

### 5.1 Introduction

The objective of this work was to explore the extrusion temperature range and screw speeds that would enable the production of a thin, rapidly disintegrating calendared film.

An important aspect and direction of these examples includes maintaining and improving the taste-masking of the API, Dextromethorphan HBr, in the film. Side-stuffing is the addition of the API-containing granules into the extruder, which happens shortly before the die. At the point of the API addition, the other formulation components, having traveled through the entire length of the extruder, have been heated, melted or softened and mixed with one another to prepare a homogeneous matrix. The side-stuffing port is just far enough from the die to allow homogeneous mixing of the API in the prepared matrix, but close enough to the die to reduce the exposure time to elevated temperatures.

For the present illustration, the method of API addition is of interest because it is assumed that exposure to elevated temperatures can affect the coating on the drug-containing granules, and thus interfere with taste-masking.

The films are evaluated by their appearance and their disintegration time. To maintain taste-masking, the amount of free drug in the film should be minimized.

### 5.2 Materials and Methods

Formulations are listed in Table 14. To make the powder blends, dry materials were weighed into a plastic bag, and mixed by shaking. The liquid component was introduced to the powder using a high-shear granulator (Robot Coupe), before loading the blend into the extruder's gravimetric feeder.

A Leistritz ZSE 18 HP twin-screw extruder equipped with a K-tron gravimetric feeder and a film die was used to extrude the formulations. Table 15 lists the extruder operating parameters for the different trials (feed rate of hopper and side-stuffer, screw speed, extruder and melt temperatures).

For film evaluations, tackiness, flexibility and brittleness were noted.

For disintegration testing, a punch and mallet were used to obtain samples of uniform size, and to standardize the handling of films. The thickness of the punched samples was measured by digital calipers (Mitutoyo), and the samples were slid into paper clips used as sinkers for the test. The disintegration test was performed on a USP disintegration tester (PharmaAlliance), in de-ionized water at 37.2° C.±0.5° C. (n=3).

TABLE 14
Formulation compositions (identical to Ex. 26).

Ex. No.

| Components, wt % | 36 | 37 |
|---|---|---|
| Coated API | 30 | side-stuffed |
| | | 30 |
| Polyox N10 | 30 | 30 |
| Mannitol | 30 | 30 |
| PEG 400 | 10 | 10 |

### 5.3 Results

Starting from the process conditions used in earlier illustrations, extrusion temperature and screw speed were first increased, and then subsequently decreased to study the possibility of milder extrusion conditions. This is relevant as stress on the coated granules is expected to release some of the coated drug, compromising the taste-masking effect. The lowest extrusion temperature found was surprisingly 50° C. (set point), with a melt temperature of 59° C. In addition, the speed of the sheet take-off rolls were varied, and a faster take-off speed resulted in a thinner film.

TABLE 15
Extrusion parameters for Example 36

| Ex. 36 | | Trial | | | | | | |
|---|---|---|---|---|---|---|---|---|
| Extrusion Parameters | Unit | 1 | 2 | 3 | 4 | 5 | 6 | 7 |
| Flow rate | kg/hr | 1.0 | 1.0 | 1.0 | 1.0 | 1.0 | 1.0 | 1.0 |
| Screw Speed | RPM | 75 | 125 | 175 | 55 | 55 | 55 | 55 |
| Melt Pressure* | PSI | 913 | 511 | 305 | 1080 | 1307 | 1418 | 1396 |
| Melt Temperature | ° C. | 79 | 91 | 107 | 71 | 68 | 63 | 59 |
| Film Label | ° C./RPM | 75/75 | 90/125 | 100/175 | 65/55 | 60/55 | 55/55 | 50/55 |

*Pressure cut-off for extruder is about 2400 PSI.

TABLE 16
Extrusion parameters for Example 37

| Ex. 37 | | Trial | | |
|---|---|---|---|---|
| Extrusion Parameters | Unit | 1 | 2 | 3 |
| Flow rate Hopper | kg/hr | 0.7 | 0.7 | 0.7 |
| Flow Rate Side-Stuffer | kg/hr | 0.3 | 0.3 | 0.3 |
| Screw Speed | RPM | 125 | 75 | 75 |
| Melt Pressure* | PSI | 542 | 764 | 1455 |
| Melt Temperature | ° C. | 97 | 86 | 60 |

*Pressure cut-off for extruder is about 2400 PSI.
(API side-stuffed)

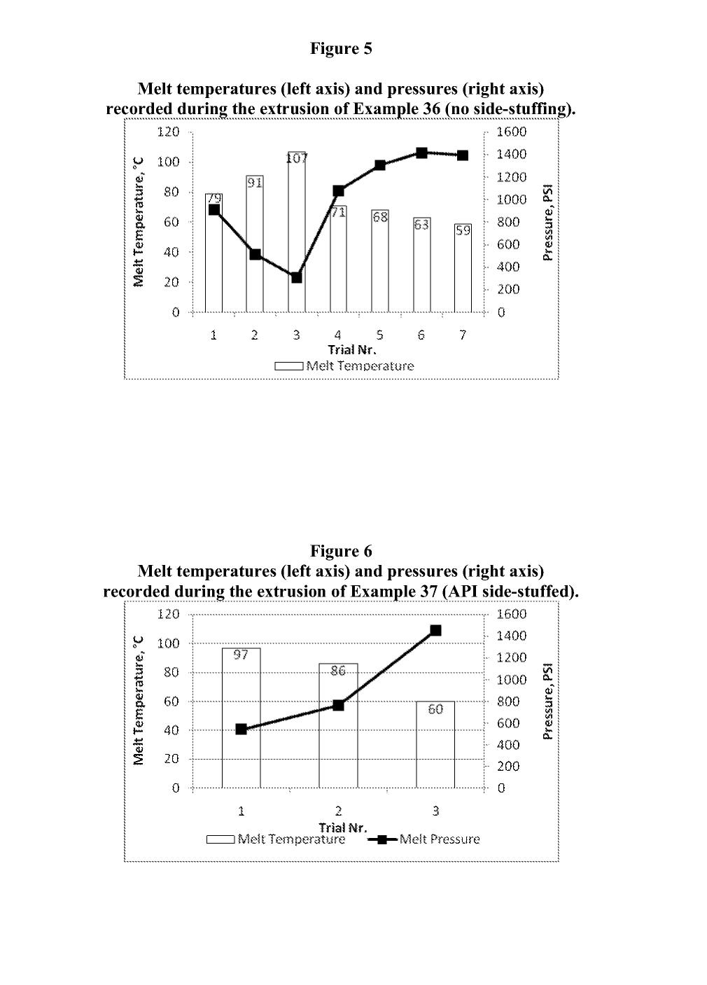

FIGS. 5 and 6 show melt temperatures and melt pressures recorded during extrusions. The melt viscosity decreases as the melt temperature increased, resulting in the inverse relation between the parameters. Film appearance was not affected by the higher melt pressures.

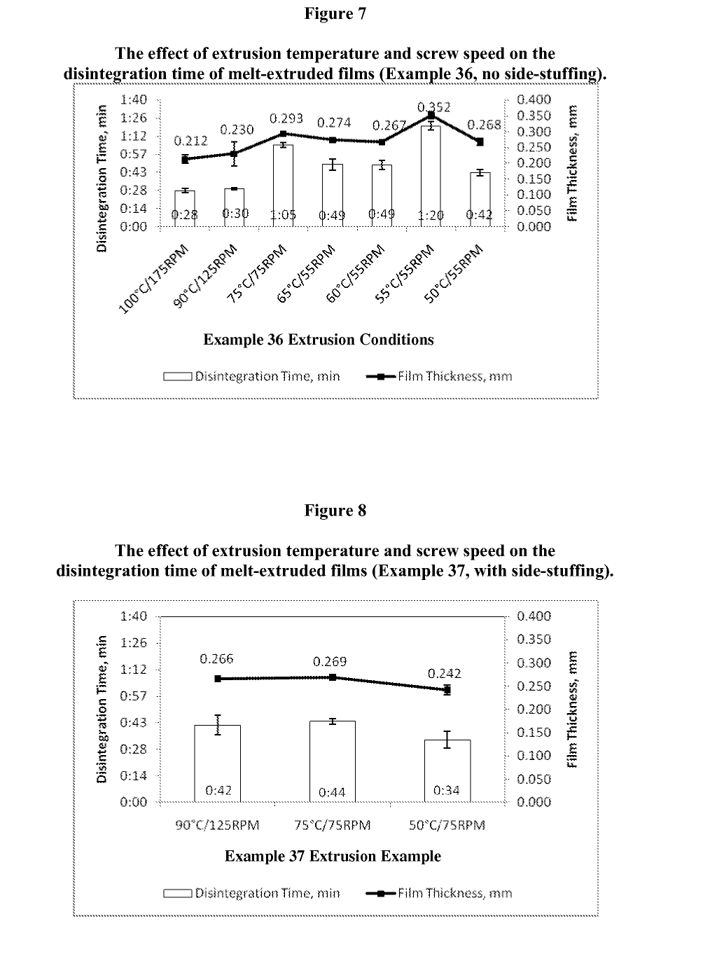

Film disintegration times were determined to understand the effect of extrusion conditions on film properties. This is a complicated analysis because of the varying film thicknesses produced under the different extrusion conditions, see FIG. 7 and FIG. 8. In the film of Example 36 (no side-stuffing), film thickness tends to increase as the extrusion temperature is decreased, resulting in thicker films, and longer disintegration times.

### 5.4 Additional Film Properties

Film stability and taste masking were also investigated. Films were placed on stability conditions (40° C./75% relative humidity and 30° C./65% relative humidity in closed laminate pouches) for 1, 2 and 3 months. Results from this study are presented in the last Illustration.

The quantity of free drug in the films are presented in the next Illustration.

### 5.5 Summary

PEO-containing thin films were surprisingly able to be extruded at lower temperatures (50-60° C.) than films of previous examples (e.g. starches). This surprising result indicates that films may be formed at temperatures well below the melting point of the coating material of the API and under conditions that will prevent leaching of free API into the matrix of the strip.

### 6 Effect of Processing Temperature and Screw Speed on the Amount of Free Dextromethorphan HBr in Melt-Extruded Films Containing Coated API Granules

### 6.1 Introduction

The purpose of the study is to detect free Dextromethorphan HBr in melt-extruded thin films in order to study the effect of the extrusion processing parameters (e.g. temperature and screw speed) on the amount of free API in melt extruded films.

The test is based on the assumption that API is released from coated, drug-containing granules during extrusion due to elevated temperature and shear experienced by the formulation. Dextromethorphan HBr is added to the film in coated form to effect taste-masking, and the presence of free drug has a negative impact on the taste of the film.

### 6.2 Principle

The test will rely on the fact that free Dextromethorphan HBr dissolves faster than API located in coated drug granules, because the coating on the granules presents an additional diffusional barrier. Test conditions were chosen such that the barrier function of the granules is enhanced.

The films were placed in a medium that dissolved the API, but retarded dissolution of the granule coating. The sample-containing vial was agitated for 2 minutes at 300 RPM in a shaker, which allowed free drug to be dissolved in the medium. For each tested sample, the amount of drug in the medium was determined by high performance liquid chromatography (HPLC) using known methods. The amount of API released from unprocessed granules during the exposure time (2 minutes) under the test conditions was quantified, and presents the baseline against which the test results from extruded films were compared.

### 6.3 Test Conditions and Set-Up

Film samples were punched from melt-extruded films using a strike die provided by the client (32×22 mm). Films were slid into paper clips, which were used as sinkers, and to provide uniform exposure of films to the medium. The test conditions are listed in Table 17.

TABLE 17
Test conditions to determine free drug in films.

| Test condition | Specification |
|---|---|
| Test vessel | 40 mL clear glass scintillation vial |
| Volume | 20 mL |
| Medium | pH 7.4 phosphate buffer USP, 50 mM KH₂PO₄ |
| Agitation | Shaker, 300 RPM |
| Sampling Time | After 2 minutes of agitation. |
| Sample volume | 3 mL |
| Sample Filtration | 10 micron free-flow filter |
| Sampling method | Manual |

Drug release from unprocessed, coated granules was determined in 12 samples after shaking at 300 RPM for 2 minutes. Unprocessed, coated granules released 2.3%±0.1 (SD) Dextromethorphan HBr in 2 minutes. All media samples were analyzed for Dextromethorphan HBr content by HPLC.

### 6.4 Test of Melt-Extruded Films

Films of the composition of Example 26 have been shown to have the most desirable properties and the unexpected ability to be processed at low temperatures. This composition has been extruded at a variety of temperatures, screw speeds and feed rates (Table 20). These films have been investigated for the Dextromethorphan HBr content using the test detailed above to study the influence of the processing conditions on the free drug content film. In addition, other films were investigated to study the effect of formulation on the free drug content (Table 18).

TABLE 18
Formulation compositions of PEO-containing films.

Composition

| | A | |
|---|---|---|
| | (identical to | |
| Components, wt % | Example 26) | B |
| Coated API | 30% | 25% |
| Polyox N10 (PEO) | 30% | 40% |
| Mannitol | 30% | 25% |
| PEG 400 | 10% | 10% |

TABLE 19
Formulation compositions of films containing hydroxypropyl starch.

Composition

| Components, wt % | C | D |
|---|---|---|
| Coated Dex | 40% | 40% |
| Lycoat RS 720 | 25% | 30% |
| (Hydroxypropyl starch) | | |
| Neosorb P110 (Sorbitol) | 10% | 10% |
| Glycerol | 7.5% | 7.5% |
| Talc | 5% | 5% |
| PEG 3350 | 12.5% | 7.5% |

As the film is immersed in the medium, Dextromethorphan HBr dissolves into it from two sources: from the drug-containing granules, and from the pool of drug outside those granules (free drug). After quantification of the API in the medium, the amount of free drug can be determined by subtracting the amount released from unprocessed granules under test conditions on average (2.3%) from the total amount of drug found in the medium.

### 6.4.1 Processing Temperature

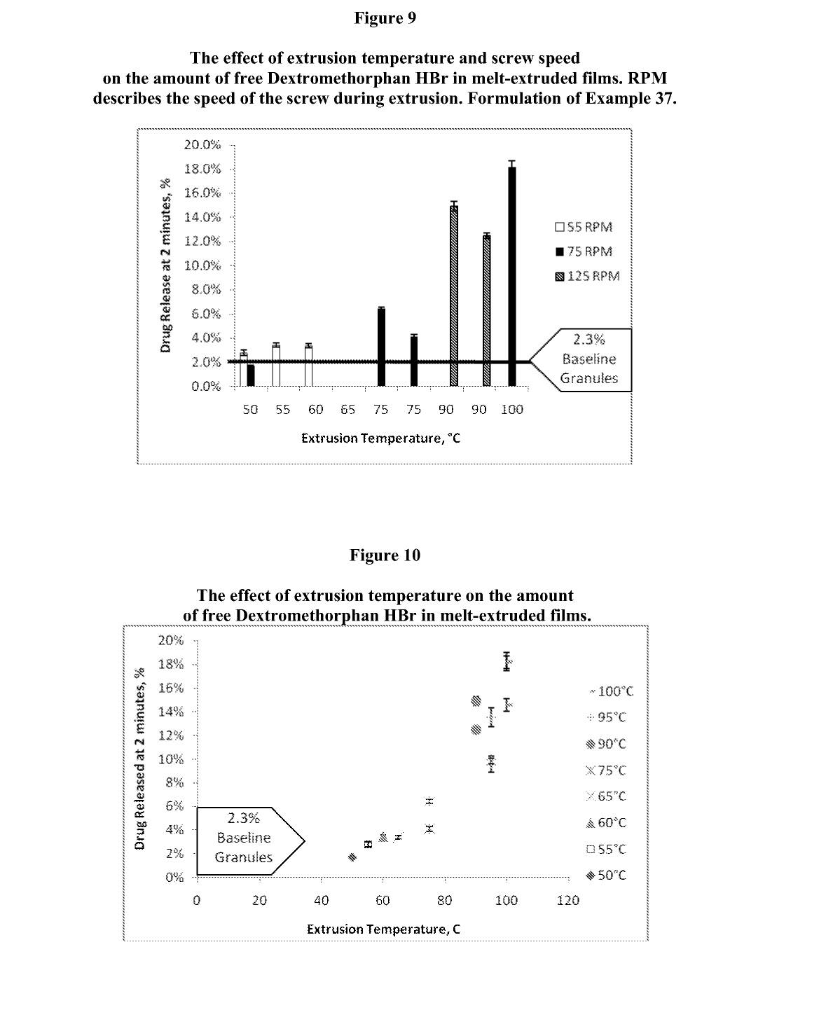

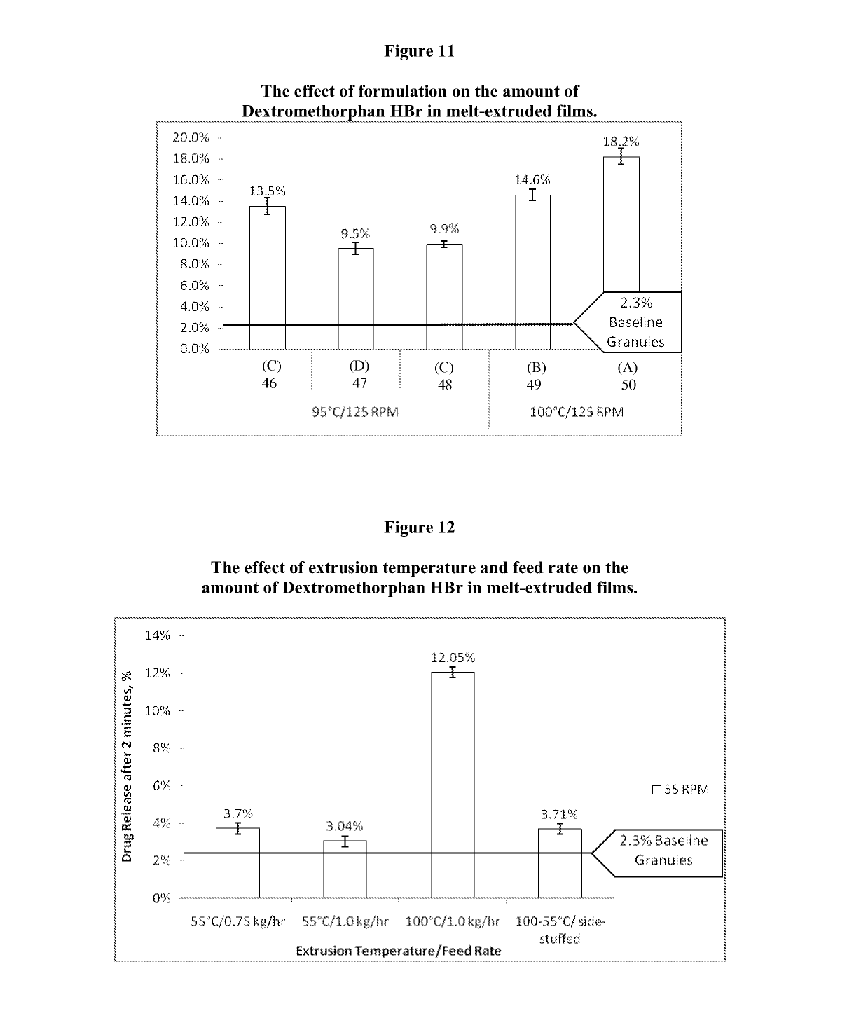

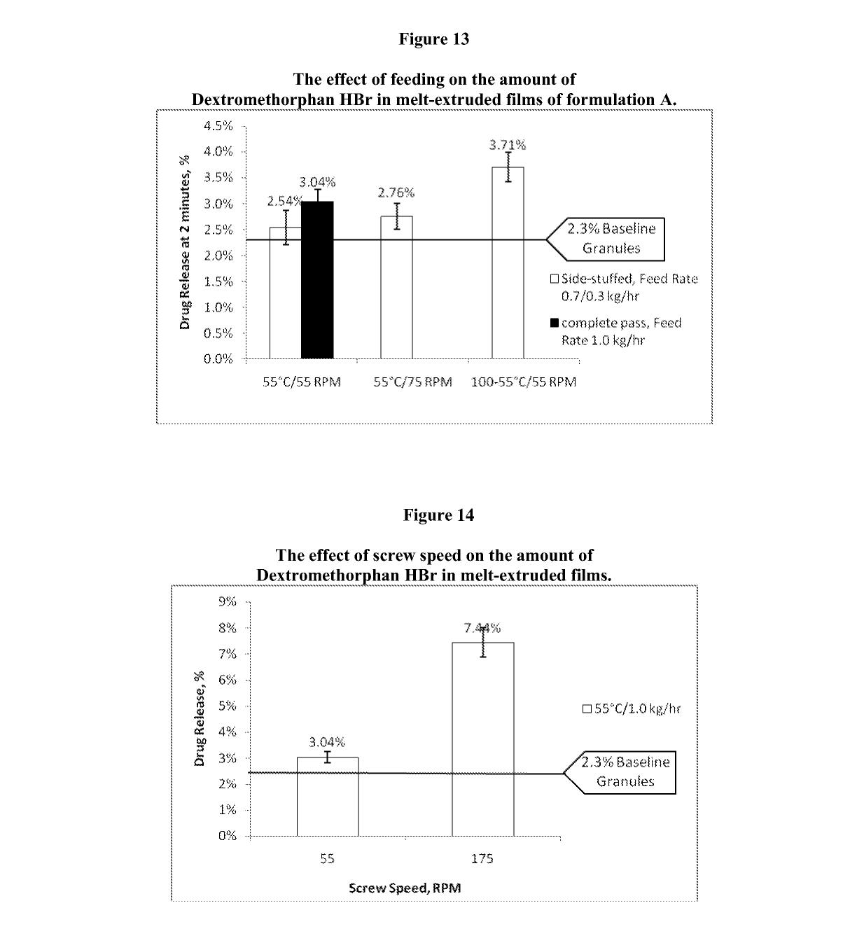

For a single formulation, FIG. 9 shows the effects of extrusion temperature and screw speed on the amount of free drug as found using the test as outlined above, and a line indicates the release from unprocessed granules, which served as a control value. API in excess of the line represents drug released from the drug granules due to melt extrusion processing.

There is a clear effect of extrusion temperature on the amount of drug found in the films of the type of compound A. The correlation remained apparent when other formulations (B-D)w ere added to the graph (FIG. 10, under different extrusion conditions shown in Table 20). Each data point represents six individual values.

Temperature can contribute to drug release if the integrity of the granule coating is impaired by exposure to elevated temperatures. Eudragit E, a component of the coating, has a glass transition of about 50-54° C. At higher temperatures under shear, the polymer can be displaced from the surface of granules, and Dextromethorphan HBr can be released through the damaged coating in larger quantities than from unprocessed granules with intact coating.

These results clearly show the advantage of being able to extrude PEO compositions at the surprising low temperatures (e.g. less than 100° C., preferably between 50-60° C.).

TABLE 20
Processing conditions of the film lots used in FIG. 10.
Example No., Processing Conditions*

38\hat{ }, 50° C./75 rpm
39, 55° C./55 rpm
40, 60° C./55 rpm
41, 65° C./55 rpm
42, 75° C./75 rpm
43\hat{ }, 75° C./75 rpm
44\hat{ }, 90° C./125 rpm
45, 90° C./125 rpm
46, 95° C./125 rpm
47, 95/125 rpm
48, 95/125 rpm
49, 100° C./125 rpm
50, 100° C./125 rpm
51, 100° C./175 rpm
*= Extrusion Temperature/Screw Speed
\hat{ }= The API of this formulation was side stuffed into the extruder

### 6.4.2 Formulation Composition

Several films with different compositions, both PEO and starch-containing formulations, were tested and compared for free API to determine the effect of formulation on free drug content. FIG. 11 shows that the formulation had a minor effect on the results, and supported the finding that processing conditions were the main influence on the release of Dextromethorphan HBR from granules during extrusion.

### 6.4.3 Feed Rate and Mode of Feeding

For a given temperature, the effect of feed rate on drug release from granules was small, as depicted in FIG. 12. Feed rates cannot be independently set, as the flow of powder from the hopper and the screw speed of the extruder have to be coordinated for a high-quality output. The same figure shows again the large effect of extrusion temperature under otherwise identical process conditions.

The addition of one component into the prepared melt at a point further down the barrel is called side-stuffing. Side-stuffed material only passes through part of the barrel, depending on the location of the port. In this case, the active was added to the prepared melt of the matrix close to the die (e.g. the end of the extruder), which reduced the exposure of the API to the melt-extrusion process. The advantage of side-stuffing includes of the opportunity to prepare and mix the matrix without damaging a thermo-sensitive active. Where process conditions are elevated or extreme side-stuffing could be advantageous to prevent thermal degradation of the API. However, the remaining components (e.g. the PEO, the plasticizer, and/or the sugar alcohol) in the composition will be exposed to the elevated or extreme conditions thereby potentially causing degradation to these components.

Examples 52 to 55 shown in FIG. 12 are based on composition A of Table 18 and are capable of being extruded with acceptable properties at the low temperature of 55° C. These examples again show that the free API in the formulation is greater where the composition is extruded at an elevated temperature. Example 55 shows a potential side-stuffing scheme where the API is introduced to the extruder after the remaining components have been first combined, heated, and mixed at 100° C., and then cooled to 55° C. This demonstrates that there is not a substantial advantage to side stuffing if processing temperatures over the length of the extruder are low (FIG. 13). The increase in amount of free drug in side-stuffed example 55 may be due to an elevated melt temperature coming down from an earlier set point temperature.

### 6.4.4 Screw Speed

Under otherwise identical processing conditions, increasing the screw speed of the extruder resulted in a larger amount of free drug in the film (FIG. 14) based upon formulation A. A higher screw speed exerted more shear on the granules, which could disrupt the coating polymer around the granule, and thus promoted drug release. (Examples 53 and 58) Formulation A extruded at 55° C. at a feed rate of 1.0 kg/hr, not side stuffed.

### 6.4.5 Summary

Taste-masking of Dextromethorphan HBr is tied to the amount of free drug in melt-extruded films, as opposed to API contained in coated granules. Extrusion temperature had a large effect on the amount of free drug detected in melt-extruded films containing of Dextromethorphan HBr. The influence of screw speed and formulation composition was smaller. Low free drug content in the films was the result of the unexpected ability to extrude the PEO containing composition at low extrusion temperatures (50-60° C.). For the formulation identical to Example 26, an acceptable film is shown to be extruded at 55° C., at a screw speed of 55 RPM, with a 0.75 or 1.0 kg/hr feed rate. These films have just over 3% free drug under the test conditions, compared with an average of 2.3% in unprocessed granules. At high extrusion temperatures (temperature gradient from 100 to 55° C.), side-stuffing of the API decreased free drug content, while it made a minimal difference at 55° C.

### 7 Extrusions of Flavored Films

The objective of this study was to extrude films containing a cherry flavor and sweetener (Sucralose), and to characterize the films for free drug content, film properties, potency and dose per unit area.

### 7.1 Film Composition and Process Conditions

The flavor active was contained in either granules (Granuseal, G), spray-dried powders (SD), Flavorburst powders (B), or in liquid form (L). All flavors contained either a high or a low amount of active flavor in a carrier, which differed between flavor formulations. All flavors with a high flavor active content were used, although films containing Flavorburst and spray-dried powders with a low flavor active content were also extruded. Since the active flavor content varied, the other foimulation components were adjusted by decreasing the PEO content and the mannitol content by equal amounts.

TABLE 21
Cherry flavors used in this study.

SL-477-500-0 Liquid 20% active
OI-398-077-4 PureDelivery Everfresh (Flavorburst)5% active
ZH-631-728-7 PureDelivery Everfresh (Flavorburst) 15% active
SP-127-249-6 PureDelivery Everfresh (Spray Dry) 20% active
WW-200-006-0 PureDelivery Everfresh (Spray Dry) 40% active
TG-248-099-5 PureDelivery Pearl (Granuseal) 5% active
OC-465-263-0 PureDelivery Pearl (Granuseal) 15% active

TABLE 22
Film Compositions

| | Composition (%, w/w) | | | | | | |
|---|---|---|---|---|---|---|---|
| | | | PolyOx | | | | |
| | Coated | Manni- | N10 | PEG | Cherry | | Sucra- |
| | API* | tol | (PEO) | 400 | Flavor | | lose |
| 59 | 30 | 25.2 | 25.2 | 10 | 6.7 | (G15%) | 3 |
| 60 | 30 | 22.3 | 22.3 | 10 | 13.3 | (G15%) | 2 |
| 61 | 30 | 25.2 | 25.2 | 10 | 6.7 | (B15%) | 3 |
| 62A | 30 | 22.8 | 22.8 | 10 | 13.4 | (B15%) | 1 |
| 62B | | | | | | | |
| 63 | 30 | 24.5 | 24.5 | 10 | 10 | (SD20%) | 1 |
| 64A | 30 | 24.5 | 24.5 | 10 | 10 | (L20%) | 1 |
| 64B | | | | | | | |
| 65 | 30 | 9.5 | 9.5 | 10 | 40 | (B5%) | 1 |
| 66A | 30 | 27 | 27 | 10 | 5 | (L20%) | 1 |

*API: Dextromethorphan HBr granules

Table 23 lists the processing conditions for the films. Processing aimed for low extrusion temperatures to minimize API release form granules, and to restrict volatilization of flavor components.

TABLE 23
Processing conditions for melt-extruded films containing coated
Dextromethorphan HBr and cherry flavors

Process Parameters

| Ex | Extrude Temp (° C.) | Screw Speed (RPM) |
|---|---|---|
| 59 | 55 | 55 |
| 60 | 55 | 55 |
| 61 | 65 | 125 |
| 62A | 55 | 55 |
| 62B | 65 | 125 |
| 63 | 55 | 125 |
| 64A | 55 | 125 |
| 64B | 65 | 125 |
| 65 | 55 | 55 |
| 66A | 55 | 125 |

### 7.2 Levels of Free Drug in Granules

Effective taste masking is related to low levels of free drug outside the API-containing, coated granules. The flavored films were tested for their free drug content using the free-API test described above. The difference in drug release at 2 minutes from a film compared to the release from unprocessed, coated, API-containing granules was used as a measure of free API in film due to melt-extrusion processing.

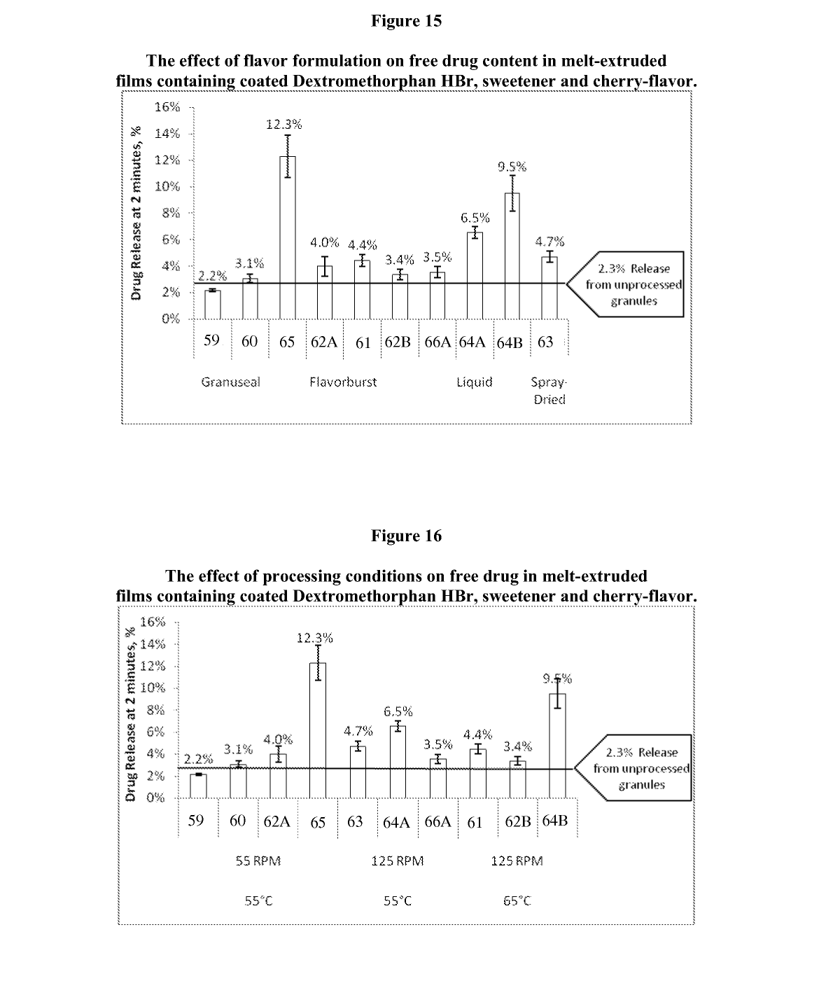

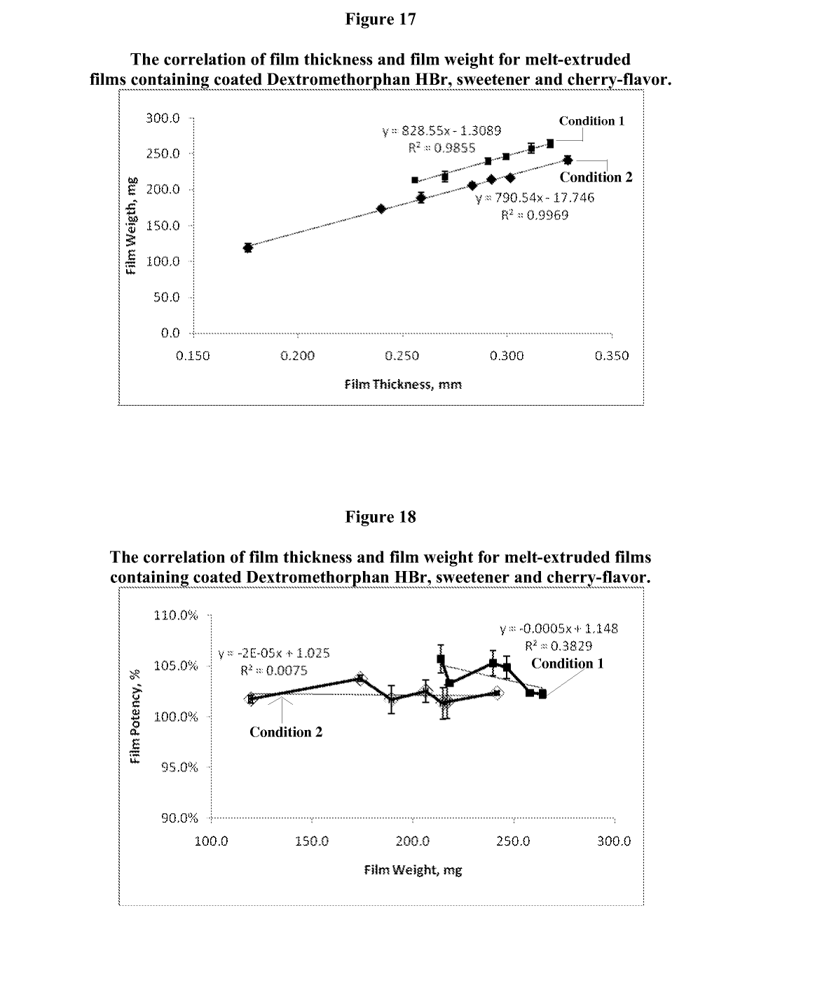

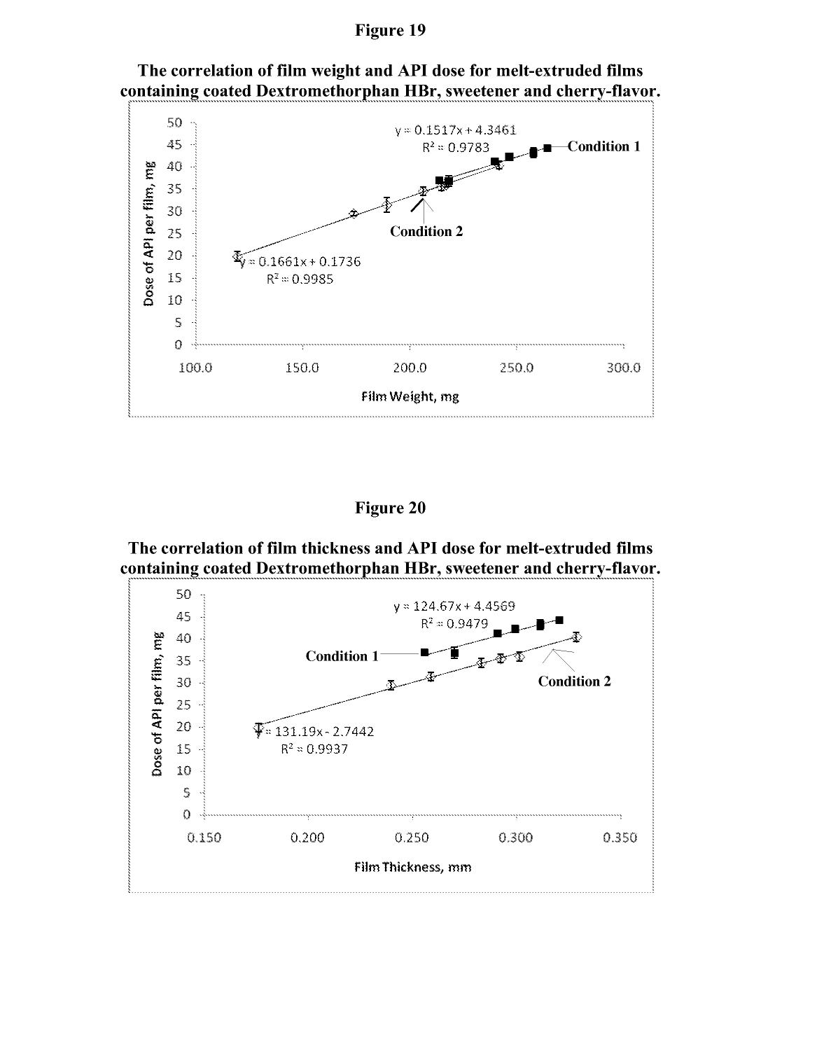

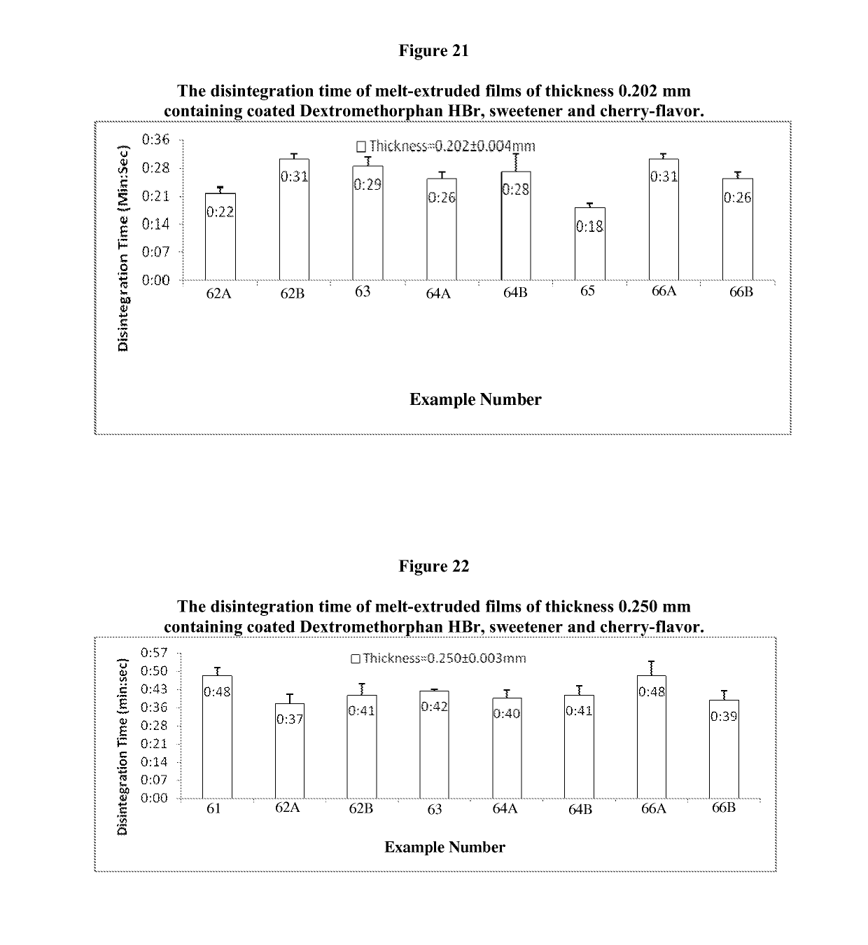

While Flavorburst powders and Granuseal granules in films resulted in lower amounts of free drug in the film, formulations containing the liquid flavor or the spray-dried powder had tended to have higher free drug values (FIG. 15, film size 32×22 mm, n=6)

An exception were films containing the Flavorburst films with 5% active flavor content, whose free drug values were very high. This could be a result of the very low polymer content of this film due to the high amount of inert carrier material for the flavor. The low amount of thermal carrier could result in higher stress on the granules during extrusion.

The high free drug of the films containing liquid flavors could be due miscibility of the lipophilic component in the flavor with the coating on the API-containing granules, which could have partially dissolved the coating and resulted in drug release.

The effects of processing conditions on free drug values are presented in FIG. 16 (film size 32×22 mm, n=6). Earlier illustrations demonstrated that free drug content increased if films were processed at higher temperatures and screw speeds. However, the effects of increasing the processing temperature from 55 to 65° C., and the screw speed from 55 to 125 RPM did not result in clear trends in free drug content. The type of flavor and processing conditions both influenced the free drug content.

### 7.3 Film Weight/Film Thickness

Two film lots (having a similar makeup to Example 61 above) were extruded using various roll speeds resulting in films with different thicknesses. Those films were analyzed for film dimensions, samples weights, potency and amount of API per dose. All film samples were cut with a strike die to a size of 32×22 mm.

Both films (e.g. condition 1 and 2) showed a strong linear correlation of weight and thickness, which indicated consistent density (FIG. 17, film size 32×22 mm, n=6).

### 7.4 Potency

Potency of films was determined to be between 101% and 106% (FIG. 18 film size 32×22 mm, n=6). The potency was independent of film weight, as indicated by the low correlation coefficient (Condition 1, R²=0.382; Condition 2, R²=0.007). This can be attributed to a constant distribution of API granules throughout the film.

### 7.5 Amount of API in the Film

In films with dimensions 32×22 mm, the API dose showed a linear correlation to the film weight (Condition 1, R2=0.998; Condition 2, R2=0.978, FIG. 19 film size 32×22 mm, n=3), and to the film thickness (Condition 1, R2=0.947; Condition 2, R2=0.993, FIG. 20 film size 32×22 mm, n=3). The close correlations again indicated a consistent film quality.

### 7.6 Disintegration Time of Films

Films of thickness 0.200, 0.250 and 0.300 mm were selected for disintegration testing. Selection of the thickness was important, as the disintegration time varies with film thickness. This was affirmed by the results in FIG. 21, FIG. 22, and FIG. 23, in which film thickness, rather than formulation, noticeably affected the disintegration time. (Film dimensions 6.5×20 mm, n=3).

### 7.7 Summary

Films containing coated Dextromethorphan HBr, Sucralose as sweetener, and cherry flavor in either granules (Granuseal, G), spray-dried (SD), Flavorburst powder (B), or in liquid form (L) were melt-extruded to investigate the impact of the addition of flavors and sweetener on melt extrusion processing, film properties and the dose of API in each film. Films containing Granuseal or Flavorburst flavors exhibited low amounts of free drug, while films with spray-dried or liquid flavors tended to show higher free drug values. However, processing at higher temperatures also increased free drug content. Film weights, thicknesses and API amounts in the film were linearly correlated, the potency was independent of film weight, which indicated even film consistency. Disintegration time of films varied with thickness, and films of 0.2 mm thickness disintegrated within about 30 seconds. Based on limited data, use of Flavorburst appears to be preferred.

### 8 Identification of Important Process Parameters for the Extrusion of Thin Films and Extrusion of 400 g and 3 kg Batches

### 8.1 Introduction

The objective of this study was to investigate the effect of excipient properties and processing parameters on the properties of thin, melt-extruded films.

Several small batches were extruded to investigate different factors and a small batch (400 g) and a large batch (3 kg) of material was extruded on the basis of these findings.

### 8.2 Formulations

All preliminary extrusions were based on the formulation in Table 24. Flavor was included only when the extrusion was determined to produce an acceptable film, that is, a sufficiently thin film. Flavor was omitted from the other extrusions to conserve the material, and was substituted in one case by inert sugar spheres (Colorcon, 25/30) to simulate their effect on the melt viscosity.

TABLE 24
Formulation of melt-extruded thin films

| | | | Film | |
|---|---|---|---|---|
| | | | Composition, % | |
| | | | Film with | Film, no |
| Component | Manufacturer | Function | Flavor | Flavor |
| Dextromethorphan | Provided | Active | 30.0 | 30.0 |
| HBr | by Novartis | | | |
| Polyethylene | Dow | Film | 22.8 | 29.5 |
| oxide (PEO) | | former | | |
| Mannitol | Roquette | Filler | 22.8 | 29.5 |
| Sucralose, | Provided | Sweetener | 1.0 | 1.0 |
| micronized | by Novartis | | | |
| Polyethylene | Dow | Plasticizer | 10.0 | 10.0 |
| glycol 400 | | | | |
| Flavor | Givaudan | Flavor | 13.4 | — |

The grades of the materials were as follows: PEO: Polyox WSR N10; Mannitol: Pearlitol 50C or 160C; PEG: Carbowax Sentry 400, Flavor: PureDelivery Pearl Granuseal Cherry flavor.

### 8.3 Results of Preliminary Extrusions

### 8.3.1 Effect of Excipient Properties

Mannitol. Two grades of Pearlitol were used in the extrusions with particle diameters of 50 and 160 micron, respectively. Both grades were screened before use (60 mesh stainless steel screen). Particle size was determined to have no effect on the ability of the blend to be extruded into thin films under the extrusion conditions.

PEO. Water is considered to be an excellent plasticizer. Only a single batch of PEO was used, but the material was dispensed several times, and moisture pick-up was considered. LOD determination of three bags (moisture balance, heating of 3 grams to 105° C.) yielded moisture values lower than 1% for all samples. Product literature indicates that PEO hygroscopicity is low, and moisture levels remains well below 3% up to relative humidity levels of 70% to 80%.

To study the effect of elevated moisture in the powder, 2 wt % DI water were added to PEO by high shear granulation, and the blend was prepared by granulation in the high shear granulator. The extrusion conditions did not change, and no obvious effect on the film was observed in the limited time immediately after extrusion.

Silicon dioxide. Without the addition of flow aids, flow properties of the granulated blend were poor, and resulted in hopper build-up. The addition of 2% colloidal silicon dioxide (Sipernat 160PQ, Evonik) to the granulated blend improved the blends flow properties to the point that no manual agitation was required. However, in an earlier illustration, the addition of colloidal silicon dioxide prolonged the disintegration time of the thin film.

### 8.3.2 Addition of Materials to the Extruder

For initial studies, formulation components varied between extrusions, and batches were small (about 300 g). All solid formulation components were blended together in a plastic bag, and the powder blend was transferred to a high shear granulator (Robo Coup), were additional mixing and the addition of liquid components (“granulation”) occurred.

This blend was not free flowing, and required manual agitation in the extruder hopper to prevent wall build-up and bridging. A constant material input was required to achieve a constant output of the extruder; hence constant powder feeding was critical. Manual agitation resulted in surging, which manifested itself in fluctuating film width.

Granulation of PEG 400 and PEO, followed by the addition of the remaining powdered components improved flow, and reduced build-up in the hopper.

The hot-melt extruder can be configured to accept several material feeds. The liquid component (PEG 400) can be added by injection into the barrel directly, metered by a peristaltic pump (Flowcon 1003), eliminating the need to granulate it with other powder components. The active can be added by an additional feeder (feeder 2) downstream, close to the die, which reduces the exposure of the active to elevated temperatures. The remaining powder components were blended in a plastic bag, and added to the main feeder (feeder 1). Splitting the feed streams accomplished several goals. It eliminated the granulation step, improved the powder flow properties of the powder blend, and reduced the temperature load on the active. The material addition remains flexible, and can be adjusted for additional process optimization. Feeding in this manner was used for the extrusion of the 400 gram batch.

### 8.3.3 Use of Gear Pump

Maintaining an even film appearance throughout extrusion can ensure a consistent product. To eliminate inevitable small fluctuations in the extruder output, and to assure consistent material flow into the die, a gear pump was installed inline between the end of the barrel and the die.

A gear pump is a positive displacement pump that precisely meters the melt to the die, and that can build and maintain a constant output pressure. It can buffer inevitable small variations in material inflow and input pressure of the extruder.

### 8.3.4 Die Gap Setting, Calendar Temperature and Gap Setting

The melt was shaped into a thin film by extrusion through a film die, in which the melt flows though a wide, thin gap, followed by calendaring, in which the film is squeezed between two temperature-controlled, rotating rolls. When the calendar temperature was too low (e.g. chilled to 15° C., or not temperature-controlled at all), the films were difficult to stretch resulting in thicker films. The optimal temperature was found to be 30° C. to 35° C., as films stuck to the roll when it was set to 50° C., and stretching became harder below 35° C.

The gap between the calendaring rolls was the last influence in shaping the film before it cools into solid form, which made it an important parameter. The gap setting was smaller than the desired film thickness, since the melt was elastic, and swelled after emerging from the rolls.

To extrude films of the desired thickness, both the die gap and the calendar roll gap settings were important. The thickness of the die gap, however, also impacts the extruder output. The extruder output decreased when the die gap was smaller, since the exit was restricted. When the die gap was small (0.2 mm), output was so low that the material backed up, and caused pressure spikes. Screw RPM and gear pump speed could not be set low enough to address the issue (decreasing material flow into the die), so the die gap was widened to increase extruder output and avoid the pressure spikes, and the calendar roll gap was decreased to control the film thickness. When the die gap was too wide (about 0.9 to 1 mm), the calendaring rolls were insufficient to decrease film thickness to below 0.3 mm. This limitation is due to the small interior volume and width of the film die used in the process, and would be addressed by a larger die.

Die temperature, die gap size, extruder screw speed and gear pump speed must be coordinated to ensure proper output.

### 8.4 Extrusion of 400 g Batch

### 8.4.1 Objective

The aim of this study was to identify the film thickness which delivers 100% potency of Dextromethorphan HBr (dose: 15 mg) in a 22×22 mm film cut from the melt extruded web.

Using the parameter setting information obtained in the preceding experiments, a 400 g batch film was extruded with a range of film thicknesses. Dextromethorphan HBr potency was measured in film samples with three thicknesses (0.3 mm, 0.4 mm, 0.5 mm, n=3), and the data was plotted to determine a correlation between film potency and film thickness for a film size of 22×22 mm. The target thickness for the 3 kg batch run was selected using the correlation.

### 8.4.2 Formulation

The formulation for the 400 g batch is the described above in Table 24. The blend contained components nr. 2, 3, 4 and 5, and was prepared by mixing the powers in a plastic bag as before. The API (1) was side stuffed, and the plasticizer (6) was metered into the extruder using a peristaltic pump.

TABLE 25
Composition of the 400 g batch and the 3 kg batch.
Method of addition refers to the introduction of a material
into the melt-extrusion process.

| Nr. | Blend Component | Details | Method of addition | % wt |
|---|---|---|---|---|
| 1 | Dextromethorphane | Coated | Side-stuffed, | 30.0 |
| | HBr | granules | feeder 2 | |
| 2 | Polyox | PEO, MW | Powder blend, | 22.8 |
| | WSR N10 | 100,000 | feeder 1 | |
| 3 | Pearlitol 50C | Mannitol | Powder blend, | 22.8 |
| | | | feeder 1 | |
| 4 | Sucralose | Micronized | Powder blend, | 1.0 |
| | | | feeder 1 | |
| 5 | Granuseal | Granules, | Powder blend, | 13.4 |
| | Cherry flavor | 15% flavor* | feeder 1 | |
| 6 | Carbowax | PEG, | Liquid feed, | 10.0 |
| | Sentry 400 | MW 400 | peristaltic pump | |

*denotes active flavor components; the remaining 85% of the granules were filler

### 8.4.3 Process Parameters for 400 g Batch,

Process parameters for the melt extrusion of the 400 g batch were derived from the preceding experiments, and are listed in Table 25.

TABLE 26
Process parameters for the extrusion of the 400 g batch.

| Parameter | Unit | Value |
|---|---|---|
| Feeder #1 flow rate (powder blend) | Kg/hour | 0.758 |
| Side stuffer (Feeder #2) flow rate (API) | Kg/hour | 0.379 |
| Side stuffer (Feeder #2) feeding screw speed (API) | RPM | 100 |
| Peristaltic pump speed (PEG 400) | RPM | 04 |
| Extruder barrel temperature | ° C. | 55 |
| Extruder screw speed | RPM | 100 |
| Gear pump speed | RPM | 20 |
| Film die gap | mm | 0.80 |
| Calendar roll gap | mm | 0.15 |
| Calendar roll temperature | ° C. | 35 |

### 8.4.4 Results

The combination of 0.8 mm die gap and 0.15 mm calendar roll gap yielded films ranging from 0.3 to 0.5 in thickness. Films were light in color. Small dots in the film were due to the larger granule size of the flavor used in the formulation (these features were absent in films of identical composition without the flavor).

### 8.4.5 Potency Analysis and Correlation to Film Thickness for

After cooling overnight, film samples (22×22 mm) were cut from the web with a strike die. The content of Dextromethorphan HBr was determined in films and Dextromethorphan HBr potency was calculated based on the desired dose of 15 mg.

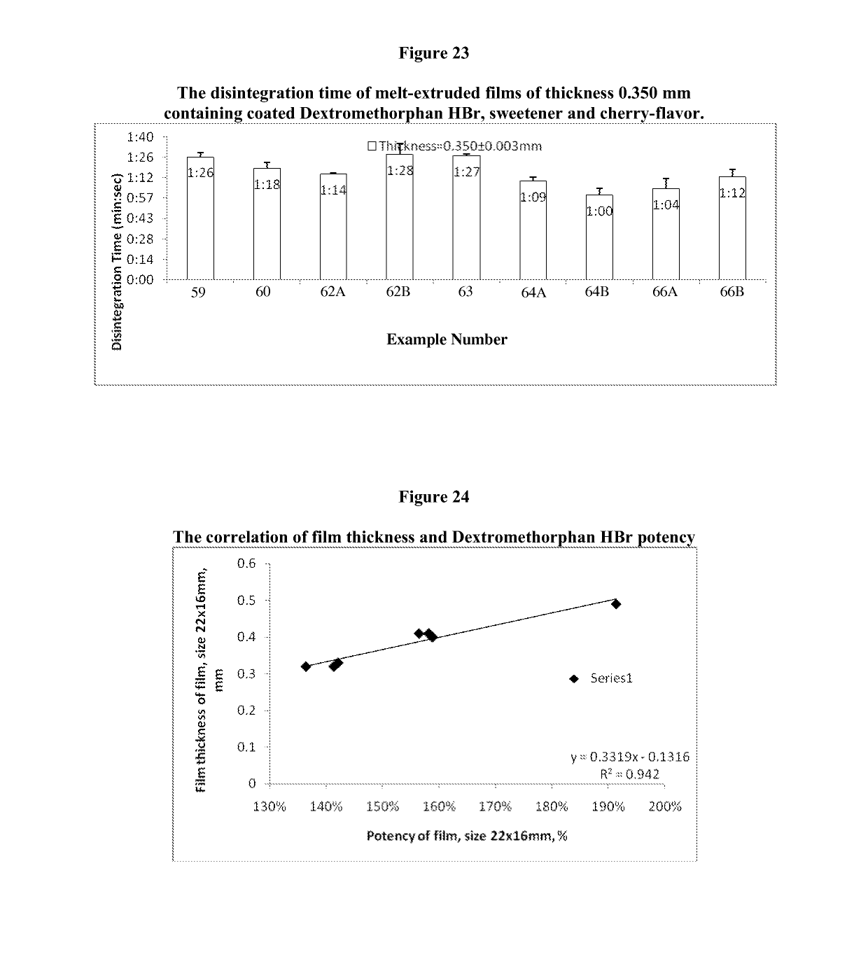

Films with a measured thickness of about 0.3, 0.4 and 0.5 mm contained on average 28.9 mg, 32.6 mg and 43.5 mg of API, respectively. Thus, potency of all films was above the desired value (FIG. 24-Potency based on a dose of 15 mg API per film sample). Using the linear correlation equation y=0.1471x+0.0636, 100% potency would be achieved in films of thickness 0.110 mm. Achieving this film thickness is unrealistic with the current equipment.

To find a combination of film size (length×width) and film thickness that yields a film of 100% potency, the potency for a smaller film size, 22×16 mm was calculated from the existing data, and those calculated potency data points were graphed to yield a linear correlation equation. Depending on which points were included, the correlation equations predicted that film thicknesses in the range of 0.13 mm (R2=0.95), 0.20 mm (R2=0.94) and 0.26 mm (R2=0.86) were required for a film 22×16. The medium film thickness of 0.2 mm was targeted.

### 8.4.6 Extrusion of Film to Set Die Gap and Calendar Gap Size

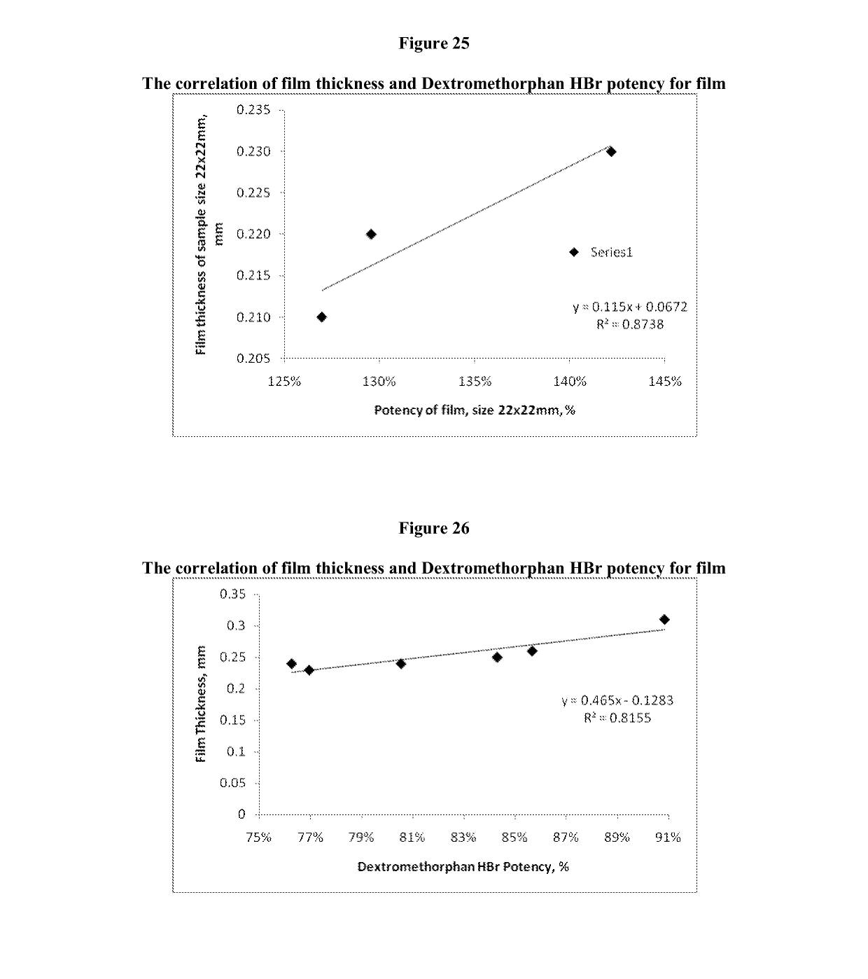

The combination of 0.8 mm die gap and 0.15 mm calendar gap used in the 400 g batch extrusion yielded films thicker than the 0.2 mm targeted for the 3 kg batch. Therefore an additional extrusion was run to determine the film thickness obtained with new settings for the die gap (0.7 mm) and the calendar gap (less than 0.1 mm). A batch containing 30% API, 30% PEO, 30% Mannitol (screened Pearlitol 160C) and 10% PEG 400 was extruded, and film thicknesses of 0.2 to 0.25 mm were obtained. Again, Dextromethorphan HBr potency was determined in films of size 22×22 (n=3). FIG. 25 shows the correlation of film thickness and Dextromethorphan HBr potency. Calculated from these values, a 22×16 mm film would have to have a thickness of about 0.23 mm for 100% potency.

### 8.5 Extrusion of the 3 kg Batch

### 8.5.1 Objective

A larger, 3 kg batch size was extruded to investigate the consistency of extrusion parameters over a longer run.

### 8.5.2 Process Parameters and Extrusion Results

Based on these results above, the 3 kg batch was extruded. Table 25 lists the composition, and Table 27 lists the process parameters. The die gap size was 0.7 mm and the calendar gap was reduced to less than 0.1 mm (smallest gauge available).

Extrusion proceeded for 2 hours and 10 minutes, and produced a thin, light-colored film. Further process optimization is necessary to match extruder screw speed, gear pump speed and die parameters for continuously steady output. Roll speed was adjusted in process to obtain a continuous film, and a low film thickness.

TABLE 27
Process parameters for the extrusion of the 3 kg batch.

| Parameter | Unit | Value |
|---|---|---|
| Feeder #1 flow rate (powder blend) | Kg/hour | 0.758 |
| Side stuffer (Feeder #2) flow rate (API) | Kg/hour | 0.379 |
| Side stuffer (Feeder #2) feeding screw speed (API) | RPM | 100 |
| Peristaltic pump speed (PEG 400) | RPM | 04 |
| Extruder barrel temperature | ° C. | 55 |
| Die temperature | ° C. | 65 |
| Extruder screw speed | RPM | 125 |
| Gear pump speed | RPM | 15-22 |
| Film die gap | mm | 0.70 |
| Calendar roll gap | mm | <0.1 |
| Calendar roll temperature | ° C. | 35 |

### 8.5.3 Analytical Characterization of the Film

Film potency, free drug content and disintegration time were determined to characterize the film.

Free Drug Content. Free drug content was determined by the test described above, and was used as a measure of how taste masking (intact barrier on API granules) was affected by melt processing. High free drug levels are associated with a decrease in taste masking. Briefly, the test measured the amount of API released into an aqueous medium after 2 minutes of agitation. The percentage of drug in excess of that released by unprocessed granules was considered to be free drug in the film released from the granules by processing and/or storage. The present films (22×16 mm, n=6) released 4.2%±0.4% (SD) API under the test conditions, which was in line with earlier results. An equal amount of unprocessed granules released 2.2%±0.15% (SD) API under the same test conditions (baseline). Processing thus increased the free drug in the film as determined by the test from about 2.2% to 4.2%. This increase can be considered small compared to films processed at higher temperatures, and therefore the taste masking should be maintained.

Disintegration time. At 37° C., films of size 22×16 mm, thickness 0.24 mm, disintegrated after 0:43±0:01 seconds in de-ionized water (n=3).

Potency. API granules were evenly dispersed throughout the film, and thus 100% potency could be achieved by changing the film size/thickness, or by adjusting the percentage of the API in the extrusion blend. The study concentrated on the former to leave the formulation unaltered. An increase in film thickness was limited, since thicker films disintegrate slower, and the desired film disintegration time is short. Film size was adjusted by cutting samples with strike dies of varying dimensions.

The goal of this characterization was to identify a film size which delivered 15 mg of Dextromethorphan hydrobromide. Based on the correlation equation in FIG. 25, it was estimated that a 22×16 mm film size would be sufficient. For films of that size, (n=6), the average film weight and thickness were 114.4 mg±11 mg (SD) and 0.255 mm±0.03 mm (SD), respectively. The average potency of these films was 82.4%±5.6% (SD) (FIG. 26), delivering on average 12.4 mg±0.8 mg API. The variation in the results was affected by the variation in film thickness of the samples.

Since the potency of the films made to these specifications was below 100%, films of size 22×18 mm were analyzed. On average (n=6), these films weighed 136 mg±3.6 mm, had a thickness of 0.27 mm±0.008 mm, and contained 13.7 mg±0.5 mg API, corresponding to a potency of 91.3%±0.04%.

Using linear correlations, a film with 100% potency should weigh between 137 mg and 142 mg, and have a thickness between 0.285 mm and 0.295 mm.

### 8.5.4 Summary

Excipient variations such as mannitol particle size or PEO moisture levels had little effect on film properties. For a given set of extrusion parameters, the method of feeding, extruder screw speed and gear pump speed, the die gap size and the calendar temperature and gap size were determined to be critical for the extrusion of thin films.

Parameters were specified that enabled the extrusion of films 0.2-0.5 mm thick, and a 400 g batch extruded under these settings. Film strips 22×22 mm delivered between 28.1 mg (187.6% potency, based on 15 mg dose) and 46.3 mg (309.0% potency, based on 15 mg dose). A correlation of potency and film thickness was used to calculate a target film thickness of 0.2-0.25 mm.

The 3 kg batch was extruded with a die gap setting of 0.7 mm and calendar gap of less than 0.1 mm. Films 22×16×0.24 mm delivered 12.4 mg API (potency of 82.4%, based on 15 mg dose), a disintegration time of 0:43±0:01 seconds, and a free drug content of 4.2%±0.4%. Films 22×16×0.27 mm contained 13.7 mg±0.5 mg API, corresponding to a potency of 91.3%±0.04.

In conclusion, melt extrusion can be utilized to produce thin films, whose characteristics (API dose, film dimensions per single dose and disintegration time) can be adjusted.

### 9 Results of a 3 Months Stability Study of Thin, Melt-Extruded Films

### 9.1 Introduction

Three initial formulations have been placed on stability in closed containers at 30° C./65% relative humidity and at 40° C./75% relative humidity (accelerated conditions) to investigate the chemical stability of the API, and the physical stability of the film. Chemical stability was assessed by Dextromethorphan HBr potency, and physical stability was characterized by measuring the disintegration time, moisture content, and the free drug content in the film.

### 9.2 Formulations

Films were stored in sealed Mylar® bags at 30° C./65% relative humidity and at 40° C./75% relative humidity (accelerated conditions). Film compositions are listed in Table 28 and Table 29.

TABLE 28
Compositions of melt-extruded films containing PEO.

| Formulation Composition | | |
|---|---|---|
| (Extrusion Temperature/ | | |
| Screw Speed) | 1 (100° C./125 RPM) | 2 (50° C./55 RPM) |
| Coated Dextromethorphan | 30% | 30% |
| HBr | | |
| PEO; Poly Ox WSR N10 | 30% | 30% |
| Mannitol; Pearlitol 50C | 30% | 30% |
| PEG 400 | 10% | 10% |

TABLE 29
Compositions of melt-extruded films containing starch

| Formulation Composition | 3 (95° C./125 RPM) |
|---|---|
| Coated Dextromethorphan HBr | 40% |
| Lycoat RS 720 | 30% |
| Neosorb P110 | 10% |
| Glycerol | 7.50% |
| Talc | 5% |
| PEG 3350 | 7.50% |

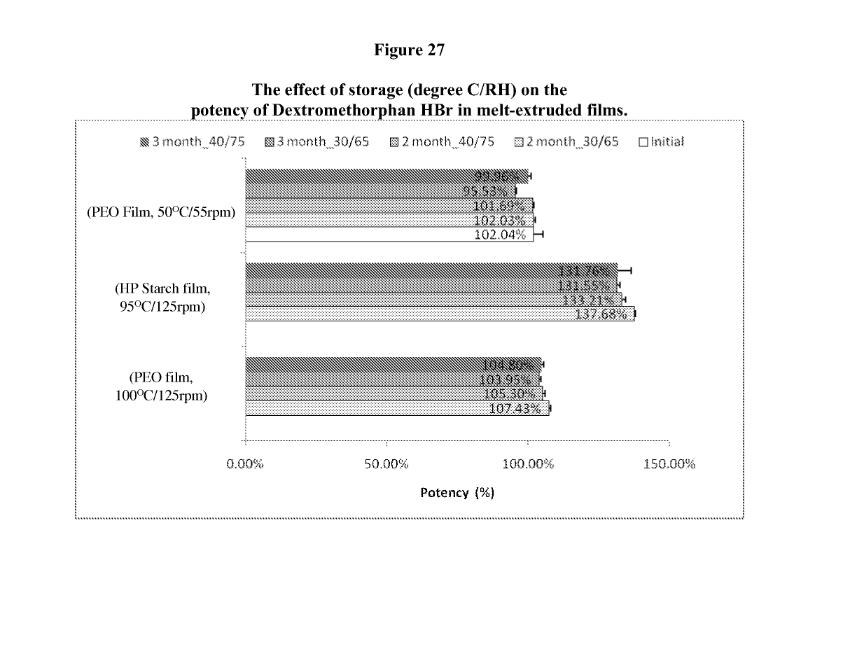

The potency of the API was determined after 2 and 3 months of storage at the conditions listed, and the data is shown in FIG. 27. Potency in all formulations showed a slight downward trend. Compositions did not contain any stabilizing components such as antioxidants.

### 9.3.2 Moisture Content/Loss on Drying

The amount of moisture in melt-extruded films was monitored to ensure the integrity of the packaging, and as an indication of the overall stability of the formulation.

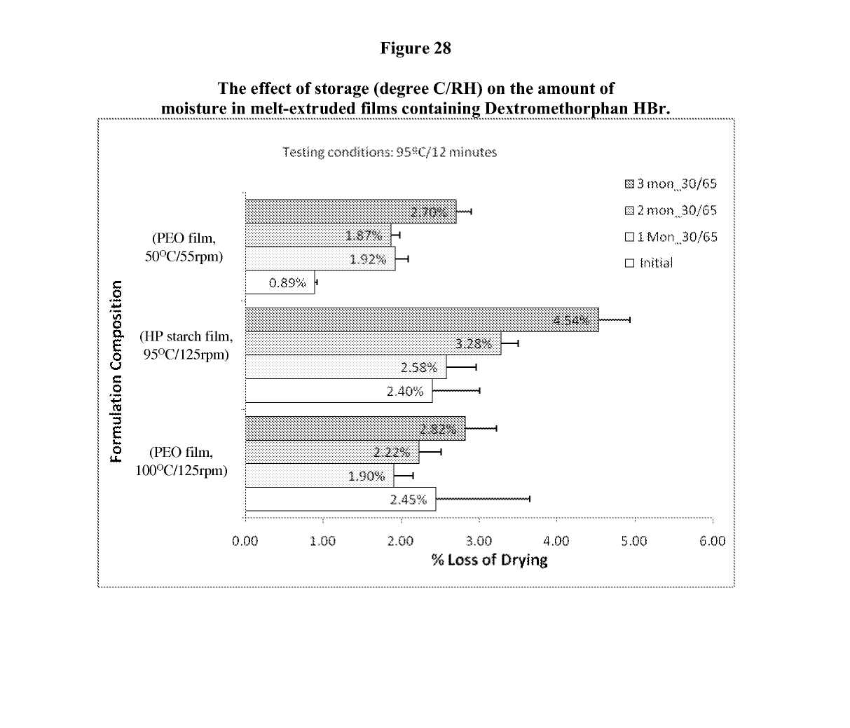

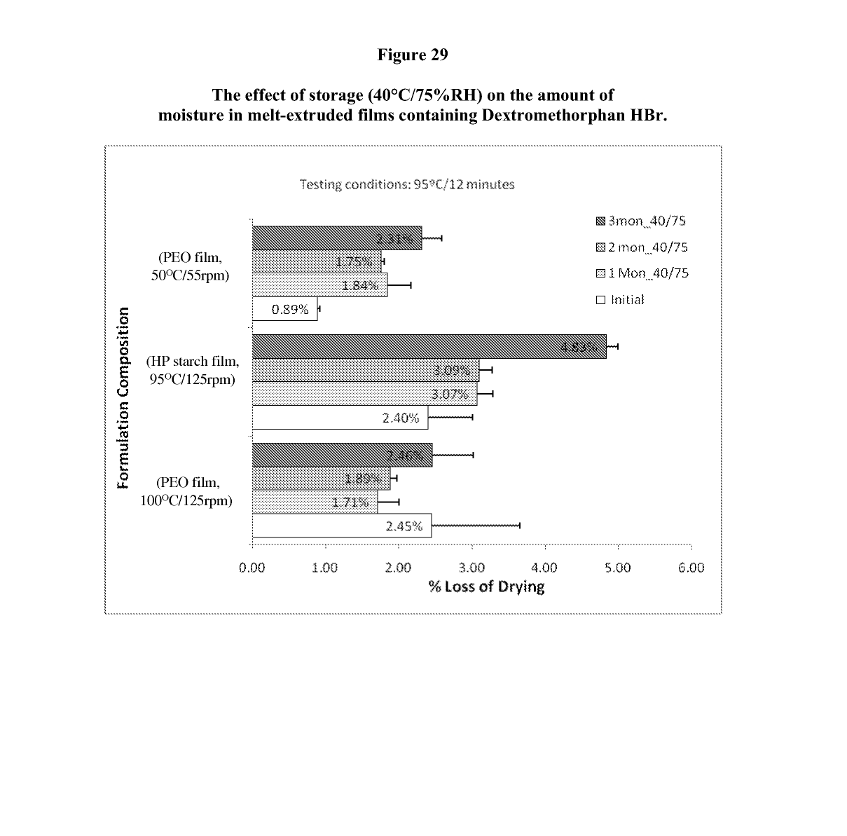

The loss on drying technique (LOD) was used to measure the moisture content of the film samples. Films (about 1 gram per test) were heated in a moisture balance at 95° C. for 12 minutes. Results are graphed in FIG. 28 and FIG. 29.

The PEO-containing formulation 2 showed an increase in moisture content from about 0.9% to over 2% in the 2-month storage period. Composition 1 moisture content remained stable in the 2.5 to 3% range. The moisture content in the starch-containing film increased from 2.4% to over 4%. Behavior of the films was similar under either storage condition.

### 9.3.3 Disintegration Time

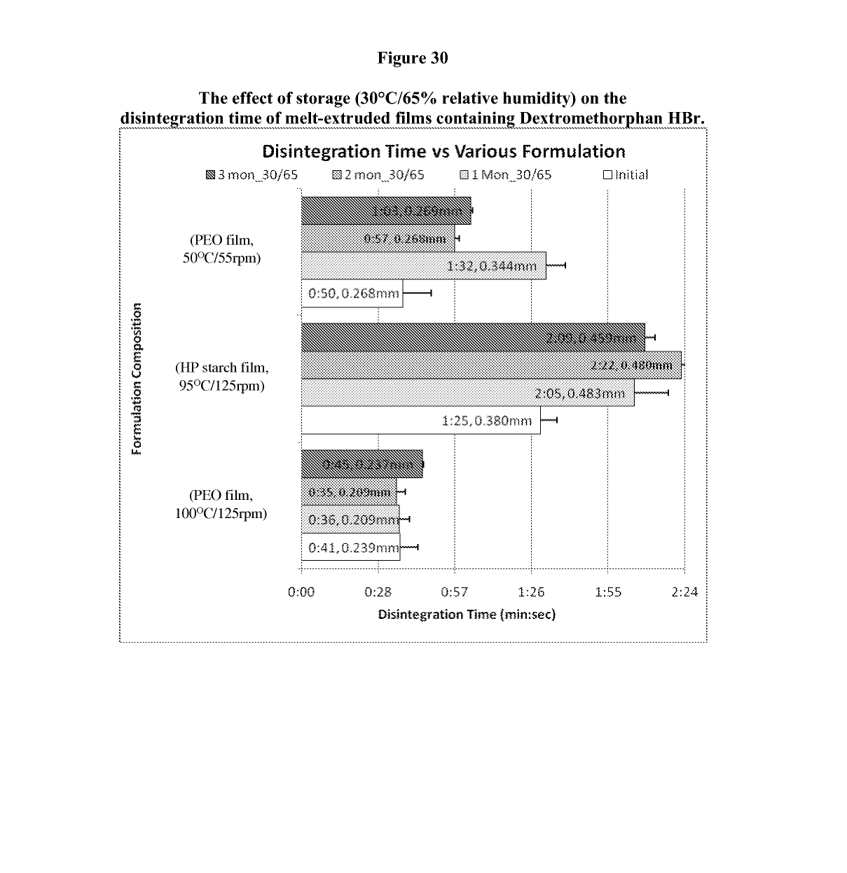

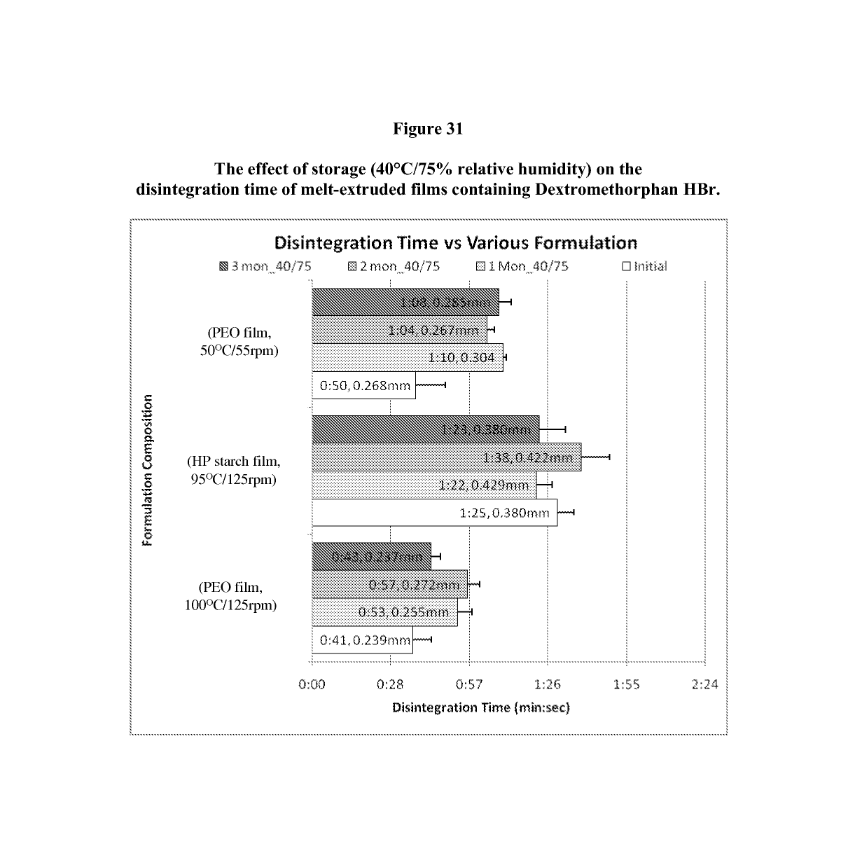

At either storage condition, disintegration time was unchanged in film 1 (FIG. 30 and FIG. 31). A small increase in the disintegration time became apparent where films of equal thickness could be compared in films 2 and 3.

Films which experienced an increase in moisture levels over storage also showed an increase in disintegration time. The relation of these two events is unclear at this time, and remains to be investigated. Water can function as a plasticizer, and may impact PEO crystallization. A semi-crystalline PEO film would be expected to have a longer disintegration time compared to a non-crystalline (amorphous) film.

### 9.3.4 Free Drug Content

“Free drug” pertains to API outside of the coated granules in the film, which can be correlated to poor taste masking, as the drug molecules would be available to the taste buds in the mouth, and would not be shielded by the granule coating. The test measured the amount of API released into an aqueous medium after 2 minutes of agitation. The percentage of drug in excess of that released by unprocessed granules (2.3%) was considered to be free drug in the film released from the granules by processing and/or storage.

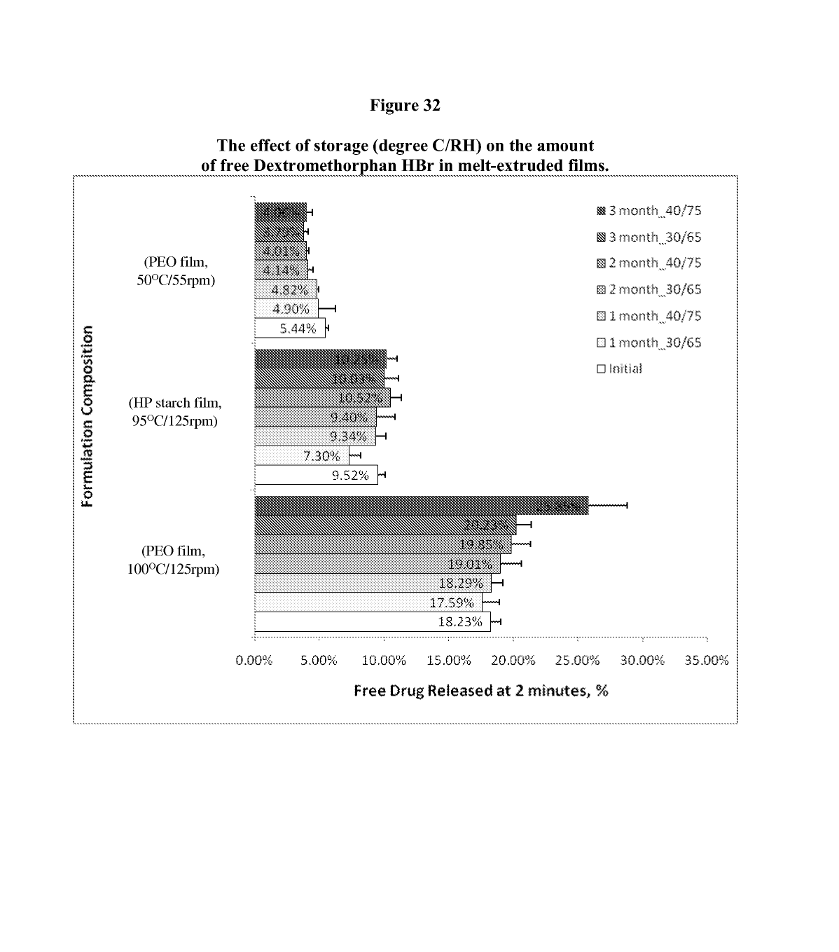

The free drug content in films is graphed in FIG. 32 (e.g. Baseline, defined as the release of API from unprocessed granules under test conditions, was 2.3%, API in excess of this value was considered free drug released by processing/storage). The preceding examples demonstrated tha processing temperatures in the range of 50 to 60° C. surprisingly resulted in low free drug values of films, which was confirmed by the results for film 2, which was processed at 55° C., and showed results in the 4-5% range. For this film, no increase in the test results was observed during the storage period, demonstrating that storage had no effect on free drug values.

Films of composition 1 were extruded at high temperatures for this study (100° C.), and consequently showed higher values of free drug in all films sampled. The free drug values increased over the 2 months storage time. Further study would be needed to confirm and evaluate the significance of this trend.

Over the 2 months of storage, the free drug content in the starch-containing film remained stable around 10%. The high processing temperature, 95° C., accounted for the higher free drug content of the film.

Overall, the results show that storage, especially at elevated temperatures, can increase the free drug content in films processed at higher temperatures. In addition, results indicate that extrusion at low temperatures not only result in low initial values for the free drug content, but that free drug content in such films remained more stable during storage.

### 9.3.5 Summary

Two PEO-containing films and one film containing hydroxypropyl starch were placed on stability at either 30° C./65% relative humidity or at 40° C./75% relative humidity in heat-sealed Mylar® bags. Initially, and after one, two and three months, the films were characterized by their disintegration time, free drug content and moisture content. In addition, potency was determined after two and three months.

Moisture content increased in films 2 and 3, and was stable in film 1. The same pattern was observed with film disintegration. The relation between the two events has not been investigated further. Free drug content was low and remained unchanged if the film was extruded at low temperatures, while extrusion at higher temperatures resulted in higher free drug values, as seen in prior work, and increased slightly over the 3 month period. Dextromethorphan HBr potency in all formulations showed a downward trend, which was slight for film 1, and larger in films 2 and 3.

### 10 Investigation on API Loadings

The objective of the present illustration is to show the drug loading variations of melt-extruded films containing API granules, and to variables to increase API content in films of a given size. Desired film properties were a high drug loading and a fast disintegration time.

### 10.1 Methods

All formulations were prepared by weighing the solid components into a plastic bag, followed by shaking to mix. The liquid component PEG 400 was added to the powder blend by high-shear mixing (RoboCoup). All formulations were extruded on a Leistritz 18 mm melt extruder, equipped with a 6-inch die (die gap was set to 0.8 or 0.6 mm). No side-stuffing was employed in this study. Films were calendared. Immediately after melt-extrusion, films were cut from the web using a strike die (22×37 mm), the films were weighed, and the films disintegration time was determined (PharmaAlliance USP disintegration tester, a larger paper clip was used as a sinker). The API content of films was calculated based on the weight of the strip (22×37 mm) and the theoretical API amount in the formulation.

### 10.2 Results

### 10.2.1 Formulations Containing Polyox

The starting point for the current study was a preferred formulation for the delivery of 15 mg Dextromethorphan HBr (API/PEO N10/Mannitol/PEG 400 in a ratio of 30/30/30/10). Changes in the formulation were based on observations made during the above extrusion. Sugar alcohol was removed from the formulation to limit the composition to three components (API/PEO N10/PEG400). At a granule loading level of 75% or greater by weight, melt viscosity became too high to form an film with preferred properties using the current equipment, and a granule content of 75% was consequently considered to be the upper limit for drug loading.

To evaluate the formulations, films were sorted into three categories, based on their disintegration time (e.g. less than 2 minutes, 2-5 minutes, and above 5 minutes). The members of the first category that disintegrated in less than 2 minutes, were ranked again by API content and by disintegration time. These two lists were compared, and two formulations were selected that ranked high on both lists (Table 30).

Table 30 shows that drug acceptable loadings of higher weight per dose drugs (e.g. Ibuprofen content of 100 mg/film; Acetaminophen content of 160 mg/film) could be achieved using the present compositions. However, the disintegration times of the current films were longer than the desired disintegration time of 30-45 seconds. Based on the foregoing examples, it is shown that adding a sugar alcohol such as mannitol will reduce the disintegration times.

TABLE 30
Formulations selected for high drug loading
levels and low disintegration times.

| | | | | | | Film | |
|---|---|---|---|---|---|---|---|
| | | | | | | Characteristics* | |
| | Content, % | | | | | DT\hat{ } | API |
| | | Polyox | Polyox | | | Aver- | Con- |
| Exam- | DXM | WSR | WSR | PEG | PEG | age, | tent, |
| ple | HBR** | N10 | N80 | 400 | 3350 | min:sec | mg |
| 10A | 45 | 45 | | 10 | | 1:33 | 152 |
| 10B | 55 | 35 | | 10 | | 1:47 | 179 |

*Film size 22 × 37 mm
**Dextromethorphan HBR granules

1. A thin strip comprising:
10 to 75% by weight of polyethylene oxide having a molecular weight of from 70,000 to 230,000 Daltons;
5 to 35% of a sugar alcohol having a melting point temperature in excess of 75° C.;
5 to 20% by weight of polyethylene glycol having a molecular weight of from 100 to 4,000 Daltons; and
5 to 75% by weight of coated active pharmaceutical ingredient (API)
wherein the thin strip is between 0.05 millimeters and 2.00 millimeters thick.

2. The thin strip of claim 1, wherein the thin strip comprises:
25 to 45% by weight of polyethylene oxide having a molecular weight of from 85,000 to 215,000 Daltons;
15 to 30% of a sugar alcohol having a melting point in excess of 100° C.;
7 to 15% by weight of polyethylene glycol having a molecular weight of from 300 to 500 Daltons; and
25 to 65% by weight of coated API.

3. The thin strip of claim 1, wherein the thin strip comprises:
30% by weight of polyethylene oxide having a molecular weight of 100,000 Daltons;
30% of a sugar alcohol having a melting point in excess of 100° C.;
10% by weight of polyethylene glycol having a molecular weight of 400 Daltons; and
30% by weight of coated API.

4. The thin strip of claim 1, wherein the thin strip further comprises between 2 and 20 wt % of a flavoring composition.

5. The thin strip of claim 1, wherein the coated API comprises an over-the-counter API selected from the group consisting of: analgesics, antihistamines, antitussives, anti-inflammatories, expectorants, upper and lower GI active ingredients, and smoking cessation active ingredients.

6. The thin strip claim 5, wherein the coated API comprises dextromethorphan hydrobromide.

7. The thin strip of claim 1, wherein the coated API is in granular form, where the average granule size is between 20 microns to 600 microns.

8. The thin strip of claim 1, wherein the coated API is in granular form, where the average granule size is between 80 microns and 200 microns.

9. The thin strip of claim 1, wherein the coated API comprises a coating selected from the group consisting of: ethyl cellulose and cellulose acetate.

10. The thin strip of claim 1, wherein the sugar alcohol comprises sorbitol, mannitol, or both sorbitol and mannitol.

11. A method of forming a thin strip according to Claim 1, the method comprising the steps of:
(I) forming a composition comprising thin strip components of the polyethylene oxide, the sugar alcohol, the polyethylene glycol, the coated active pharmaceutical ingredient (API), and optionally the flavoring composition of claim 4,
(II) melt extruding a thin sheet having to a thickness of between 0.05 millimeters and 2.00 millimeters from the composition; and
(III) cutting the thin sheet into thin strips;
wherein the processing temperature during steps (I), (II), and (III) does not exceed the melting point temperature of the sugar alcohol.

12. The method of claim 11, wherein the sugar alcohol comprises mannitol and the melt temperature during steps (I), (II), and (III) does not exceed 150° C.

13. The method of claim 12, wherein the sugar alcohol comprises sorbitol and the melt temperature during steps (I), (II), and (III) does not exceed 90° C.

14. The method of claim 13, wherein the melt temperature during steps (I), (II), and (III) is between 50° C. and 70° C.

15. The method of claim 11, wherein the thin strip is 0.1 to 0.8 millimeters thick.

16. The method of claim 11, wherein the composition is formed in an extruder during melt extrusion (II), wherein coated API is introduced to the extruder in a downstream barrel from where other thin strip components are introduced.

17. The method of claim 11, wherein the processing temperature during steps (I), (II), and (III) is below the melting point temperature of the coating of the coated API.

18. The method of claim 11, wherein the thin strip contains less than 5 times the amount of free API compared to the free API content of a corresponding amount of unprocessed coated API used in the composition.

19. The method of claim 18, wherein the thin strip contains less than 3 times the amount of free API compared to the free API content of a corresponding amount of unprocessed coated API used in the composition.

20. The method of claim 19, wherein the thin strip contains less than 1.5 times the amount of free API compared to the free API content of a corresponding amount of unprocessed coated API used in the composition.

21. The method of claims 11-20, wherein the melt extruding step (II) further includes calendering the extrudate to the thickness of between 0.05 millimeters and 2.00 millimeters, or 0.1 to 0.8 millimeters of claim 15.

22. A thin strip produced by the method of any of claim 11.
